(19) United States
(12) Patent Application Publication
(10) Pub. No.: US 2020/0188440 A1
(43) Pub. Date: Jun. 18, 2020

(54) METHODS OF INHIBITING AGING AND TREATING AGING-RELATED DISORDERS

(71) Applicant: EXOSTEM BIOTEC LTD., Tel Aviv (IL)

(72) Inventors: Chaya BRODIE, Southfield, MI (US); Aharon BRODIE, Miami Beach, FL (US)

(21) Appl. No.: 16/614,541

(22) PCT Filed: May 16, 2018
(86) PCT No.: PCT/IL2018/050538

§ 371 Date: Nov. 18, 2019

Related U.S. Application Data
(60) Provisional application No. 62/506,661, filed on May 16, 2017.

(51) Int. Cl. A61K 35/28; A61K 35/50; A61P 43/00; A61P 21/00

(52) U.S. Cl. CPC A61K 35/28; A61P 21/00; A61P 43/00; A61K 35/50

(57) ABSTRACT

Methods of treating an aging-associated disease, as well as inhibiting aging in a subject, by administering pharmaceutical compositions comprising unmodified and modified MSCs and their exosomes are provided.

### FIELD OF INVENTION

The present invention is in the field of mesenchymal stem cells (MSCs), and their use in treating aging and aging related disorders.

### BACKGROUND OF THE INVENTION

Mesenchymal stem cells (MSCs) are a heterogeneous population of mesoderm-derived stromal cells that can be obtained from autologous bone marrow, dental pulp, or adipose tissues or from allogeneic amniotic fluid, placenta and umbilical cord. MSCs exhibit minimal immunogenicity due to low levels of MHCII molecules and this characteristic is more pronounced for MSCs from amniotic fluid, chorionic placenta and umbilical cord, which are considered are non-immunogenic. Such non-immunogenic cells can be used as off-the-shelf cells as they may be administered to anyone. Exosomes form these cells are similarly non-immunogenic and may also be used in this capacity. Recent reports have demonstrated that in addition to their natural ability to differentiate to cartilage, bone and fat cells, these cells have also the potential to be trans-differentiated into other cell types, including hepatocytes, muscle, endothelial, neuronal, and insulin-producing cells.

MSCs have been shown to exert therapeutic effects in a variety of diseases and dysfunctions in experimental animal models and more recently in pilot clinical trials (Gao et al., 2015, International Journal of Cardiology, 168: 3191-3199; Zhang et al., 2013, Journal of neuroinflammation, 10:106). These cells have the capacity to migrate to and engraft in sites of inflammation and injury and to exert local effects in the resident tissues. It has been reported that the adult MSCs are non-immunogenic, which indicates that no immunosuppression is required for their transplantation into an allogeneic host.

Studies have shown that MSCs have immunosuppressive and immunoregulatory properties. The beneficial effects of MSCs have been mainly attributed to this immunomodulatory activity and the secretion of trophic factors. Indeed, MSCs secrete a large variety of bioactive molecules, such as growth factors, cytokines and chemokines and can provide trophic support to multiple tissues. In addition, recent studies demonstrated that MSCs secrete extracellular vesicles that deliver RNA and DNA molecules in addition to various proteins as a part of intercellular communication.

Use of mesenchymal stem cells (MSCs) to promote wound healing as well as support tissue growth has been known for some time. More recently it was shown that media from bone marrow and umbilical cord MSC can be used to reduce aging in skin (U.S. Pat. No. 9,284,528). However, every organ and system is effected by aging, not just the skin. As average life spans increase in developed countries due to medical breakthroughs and improvements in nutrition and lifestyle, treatments that can slow aging or treat aging-related disorders are greatly in need.

### SUMMARY OF THE INVENTION

The present invention provides methods of treating an aging-associated disease as well as inhibiting aging in a subject, by administering pharmaceutical compositions comprising MSCs and their exosomes.

According to a first aspect, there is provided a method of inhibiting aging or treating an aging-associated disease in a subject, the method comprising administering to the subject a pharmaceutical composition substantially devoid of amniotic placenta mesenchymal stem cells (MSCs), and comprising a pharmaceutically acceptable carrier and at least one of:
a. a chorionic placenta MSC;
b. exosomes from a chorionic placenta MSC;
c. a dedifferentiated MSC;
d. exosomes from a dedifferentiated MSC;
e. a differentiated MSC;
f. exosomes from a differentiated MSC and
g. a combination thereof;

thereby inhibiting aging in a subject.

According to some embodiments, the dedifferentiated MSC is produced by introducing into an MSC any one of NANOG, SOX2, KLF4, OCT4 and a combination thereof. According to some embodiments, the dedifferentiated MSC is produced by incubating an MSC in a medium containing 5-azacetidine (5-AZA). According to some embodiments, the dedifferentiated MSC is produced by further incubating the MSC in an acidic medium or in hypoxia.

According to some embodiments, the aging is selected from muscle aging, neuronal aging, pancreatic aging and joint aging. According to some embodiments, neuronal aging comprises impaired cognitive function, impaired memory or both. According to some embodiments, muscle aging comprises reduced muscle mass, increased fibrosis or both.

According to some embodiments, the aging associated disease is selected from sarcopenia, fibrosis, diabetes type 2, arthritis, muscle atrophy, Alzheimer's disease, dementia, stroke-related brain damage, and Hutchinson-Gilford Progeria Syndrome (HGPS). According to some embodiments, the fibrosis is cardiac fibrosis or skeletal muscle fibrosis. According to some embodiments, the arthritis is osteoarthritis.

According to some embodiments, inhibiting aging comprises at least one of: decreasing fibrosis, decreasing inflammation, decreasing production of reactive oxidation species (ROS), increasing muscle mass, increasing stem cell self-renewal, improving glucose homeostasis, increasing cognitive function, increasing memory, increasing chondrocyte survival and decreasing levels of progerin, SRSF1 or both. According to some embodiments, the stem cell is any one of a neuronal stem cell (NSC) and a satellite cell.

According to some embodiments, the treating comprises
a. treating an aging associated disease that is not cancer; and
b. reducing the risk of developing cancer, treating cancer or both.

According to some embodiments, the differentiated MSC is differentiated toward any one of an astrocyte, a neural stem cell, a motor neuron, an oligodendrocyte, a satellite cell and a myoblast.

According to some embodiments, the method of the invention further comprises introducing into the MSC, dedifferentiated MSC or differentiated MSC at least one regulator RNA selected from: microRNA (miR)-10b, miR-10a, miR-138, miR-145, miR-373, miR-1225, miR-375, miR-143, miR-675, long non-coding RNA (lncRNA) MEG3 and lncRNA PLUTO.

According to some embodiments, the method of the invention further comprises introducing into the MSC, dedifferentiated MSC or differentiated MSC at least one RNA inhibitory molecule that binds to and inhibits at least one of let-7, miR-424, miR-195, miR-16, miR-497, miR-135, miR-6793, miR-133b, miR-214, miR-154 and miR-21.

According to some embodiments, the subject is a human. According to some embodiments, the subject is a veterinary animal.

According to another aspect, there is provided, a genetically modified MSC, the MSC comprising exogenous microRNA let-7 and an RNA inhibitory molecule that binds to and inhibits miR-133b.

According to another aspect, there is provided, a genetically modified MSC, the MSC comprising at least one exogenous miR selected from miR-10b, miR-138, miR-145 and miR-675.

According to another aspect, there is provided, a genetically modified MSC, the MSC comprising at least one RNA inhibitory molecule that binds to and inhibits at least one of miR-424, miR-195, miR-16, miR-497, miR-135, miR-6793, miR-21 and miR-133b.

According to another aspect, there is provided, a genetically modified MSC, the MSC comprising at least one of exogenous miR-145, an RNA inhibitory molecule that binds to an inhibits miR-154 and a combination thereof.

According to another aspect, there is provided, a genetically modified MSC, the MSC comprising at least one of: exogenous miR-375, exogenous lncRNA PLUTO, an RNA inhibitory molecule that binds to and inhibits miR-21 and a combination thereof.

According to another aspect, there is provided, a genetically modified MSC, the MSC comprising at least one of: exogenous lncRNA MEG3, exogenous miR-143 and a combination thereof.

According to another aspect, there is provided, a genetically modified MSC, the MSC comprising exogenous miR-143, miR-10a, miR-373 and miR-1225.

According to another aspect, there is provided, a pharmaceutical composition, comprising
a. a genetically modified MSC of the invention; and
b. a pharmaceutically acceptable carrier, adjuvant or excipient.

Use of a pharmaceutical composition of the invention to inhibit aging or treat an aging-associated disease.

Use of a pharmaceutical composition of the invention to treat muscle aging, wherein the composition comprises a genetically modified MSC, wherein the MSC comprises at least one of:
a. an exogenous microRNA let-7 and an RNA inhibitory molecule that binds to and inhibits miR-133b;
b. at least one exogenous miR selected from miR-10b, miR-138, miR-145 and miR-675;
c. at least one RNA inhibitory molecule that binds to and inhibits at least one of miR-424, miR-195, miR-16, miR-497, miR-135, miR-6793, miR-21 and miR-133b; and
d. at least one of exogenous miR-145, an RNA inhibitory molecule that binds to an inhibits miR-154 and a combination thereof.

Use of a pharmaceutical composition of the invention to treat any one of:
a. diabetes type 2;
b. cancer or the risk of developing cancer; and
c. a combination thereof,
   wherein the composition comprises a genetical modified MSC, wherein the MSC comprises at least one of: exogenous miR-375, exogenous lncRNA PLUTO, an RNA inhibitory molecule that binds to and inhibits miR-21 and a combination thereof.

Use of a pharmaceutical composition of the invention to treat any one of:
a. arthritis;
b. neuronal aging;
c. cancer or the risk of developing cancer; and
d. a combination thereof,
   wherein the composition comprises a genetical modified MSC, wherein the MSC comprises at least one of: exogenous lncRNA MEG3, exogenous miR-143 and a combination thereof.

Use of a pharmaceutical composition of the invention to treat any one of:
a. muscle aging;
b. neuronal aging;
c. HGPS; and
d. a combination thereof,
wherein the composition comprises a genetical modified MSC, wherein the MSC comprises exogenous miR-143, miR-10a, miR-373 and miR-1225.

Use of a pharmaceutical composition of the invention to treat neuronal aging, wherein the composition comprises the genetically modified MSC, and wherein the MSC expresses an RNA inhibitory molecule that binds to and inhibits miR-21.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-C: MSCs express anti-aging factors Bar charts showing relative mRNA expression of anti-aging genes (1A) TIMP2, (1B) GDF11 and (1C) KLOTHO in MSCs from various tissues.

FIG. 2: Chorionic and umbilical cord MSC reduce ROS production A bar chart showing relative reactive oxygen species (ROS) generation 3 days after myostatin treatment.

FIGS. 3A-K: MSCs increase muscle regeneration and decrease fibrosis (3A) A bar chart showing relative expression levels of TNFα, Utrophin, Collagen I and NCAM in the quadricep muscle of MDX mice 4 weeks after injection of 5×10⁵ MSCs or their exosomes. (3B) A bar chart showing relative expression levels of Collagen I in the quadricep muscle of MDX mice 4 weeks after injection of 5×10⁵ MSCs from various tissues. (3C) A bar chart showing percent regeneration, as measured by counting NCAM positive cells in quadriceps of mdx mice 4-weeks after injection of exosomes from 5×10⁵ MSCs. (3D) A bar chart showing percent regeneration, as measured by counting NCAM positive cells in quadriceps of mdx mice 4-weeks after injection of exosomes from 5×10⁵ MSCs. (3E) A bar chart showing relative levels of utrophin expression in human muscle cells cocultured with MSCs from various tissues. (3F) A bar chart showing the % of myoblasts that had formed into myotubes of at least 4 cells, and (3G) a western blot image showing MYH2 protein expression, in healthy myoblasts after coculture with MSCs of various tissues. (3H) A bar chart showing the % of myoblasts that had formed into myotubes of at least 4 cells in myoblasts from DMD patients after coculture with MSCs of various tissues. (3I) A western blot image of MyoD protein expression in satellite cells after coculture with MSC of various tissues or their exosomes. (3J) A western blot image of MyoD protein expression in mouse C2C12 cells after coculture with MSC of various tissues or their exosomes. BM-bone marrow, AD-adipose, AM-amniotic placenta, CH-chorionic placenta, UC-umbilical cord. (3K) A bar chart showing relative fluorescence from transplanted human myoblasts or satellite cells 2 weeks after transplant. Cells were transplanted alone, or co-transplanted with MSCs.

FIG. 4: MSCs increase both proliferation and satellite cell asymmetric division in an aging muscle model A bar chart showing relative MyoD expression in satellite cell cultures that are grown in the presence of young (age 15-20) and old (age 55-60) serum. MyoD expression measures the amount of asymmetrical division of the satellite cells to myoblasts.

FIGS. 5A-B: MSCs increase NSC self-renewal (5A-B) Bar charts of NSC self-renewal after coculture in a transwell with MSCs, their vesicles, cord blood and combinations thereof, (5A) without and (5B) with treatment with hydroxyurea. Culture with just medium and without hydroxyurea was used as a control and set to 1.

FIGS. 6A-C: Use of dedifferentiated MSCs and untreated MSCs to treat sarcopenia (6A) A bar chart showing the percent change in expression of fibrosis marker Collagen I and regeneration marker NCAM in quadricep muscles of wild-type mice 4 weeks after injection of 1×10⁶ unprimed and primed MSCs. Expression levels are measured relative to a control quadricep muscle which was mock injected. (6B) A bar graph showing myotube diameter (mM) 3 days after treatment with PBS or myostatin. (6C) A bar chart showing the number of newly generated muscle fibers in the gastrocnemius muscle of wild-type mice 7 days after cardiotoxin treatment. Mice were preinjected with either PBS, MSCs or primed MSCs.

FIGS. 7A-B: Use of MSCs to treat type 2 diabetes Bar charts of blood glucose levels in diabetic mice 10 days after administration of (7A) unmodified MSCs and their extracellular vesicle or (7B) MSCs expressing miR-375 and an antagomir to miR-21.

FIGS. 8A-B: Use of MSCs to treat osteoarthritis (8A-B) Bar charts of SA beta-gal activity (senescence) in (8A) human and (8B) canine chondrocytes after treatment with IL-1beta and transwell coculture with MSCs.

FIGS. 9A-E: Use of MSCs as a therapeutic delivery system (9A) A bar chart showing relative Utrophin mRNA expression in myoblasts following incubation with exosomes from MSCs loaded with the listed antagomirs. (9B) A western blot image showing utrophin expression in muscle cells in vivo after injection of CH-MSCs expressing antagomirs to let-7 and miR-133b. (9C) A western blot image showing utrophin expression in muscle cells in vivo after injection of muscle-targeted and untargeted exosomes from CH-MSCs expressing an antagomir to let-7. (9D) A bar chart showing the relative number of myosin heavy chain positive cells after coculture with CH-MSCs expressing an antagomir to let-7. (9E) A bar chart showing the number of myoblast cells showing nuclear staining for MyoD protein following introduction into the cells of a modified MyoD mRNA by transfection, incubation with preloaded exosomes from MSCs, or trans-well coculture with MSC expressing the modified mRNA.

FIG. 10: MSCs reduce fibrosis in muscle cells A bar chart of relative Collagen 1A1 expression in skeletal muscle cells and cardiomyocytes treated with TGF-beta and transwell cultured with MSC.

FIGS. 11A-B: MSCs increase self-renewal in NSCs (11A-B) Bar charts of relative self-renewal of NSCs grown in transwell culture with MSCs (11A) without and (11B) with addition of hydroxyurea.

FIGS. 12A-B: MSCs exert anti-tumor effects on a wide variety of cancers (12A) A bar chart of the effect of MSCs and their vesicles on cancer cell proliferation. The bars represent, in order, glioma, meningioma, pancreatic, lung, prostate, breast, leukemia, lung metastasis and neuroblastoma cancer cells. (12B) A bar chart showing the decreased self-renewal of lung cancer stem cells after transwell culture with MSCs expressing MEG-3, miR-143 or a combination of the two.

Error bars provide the standard error in all figures.

### DETAILED DESCRIPTION OF THE INVENTION

The present invention provides methods of treating an aging-associated disease as well as inhibiting aging in a subject, by administering pharmaceutical compositions comprising MSCs and their exosomes.

By one aspect, the present invention concerns a method of treating an aging-associated disease in a subject in need thereof, the method comprising administering to the subject a pharmaceutical composition comprising a pharmaceutically acceptable carrier and at least one of:
a. an unmodified MSC;
b. exosomes from an unmodified MSC;
c. a dedifferentiated MSC;
d. exosomes from a dedifferentiated MSC;
e. a partially differentiated MSC;
f. exosomes from a partially differentiated MSC and
g. a combination thereof;
   thereby treating an aging-associated condition in a subject.

By another aspect there is provided a pharmaceutical composition comprising a carrier and at least one of: an unmodified MSC; exosomes from an unmodified MSC; a dedifferentiated MSC; exosomes from a dedifferentiated MSC; a partially differentiated MSC; exosomes from a partially differentiated MSC and a combination thereof; for use in treating an aging-associated disease.

By another aspect, the invention concerns a method of inhibiting aging in a subject, the method comprising administering to the subject a pharmaceutical composition comprising a pharmaceutically acceptable carrier and at least one of:
a. an unmodified MSC;
b. exosomes from an unmodified MSC;
c. a dedifferentiated MSC;
d. exosomes from a dedifferentiated MSC;
e. a partially differentiated MSC;
f. exosomes from a partially differentiated MSC and
g. a combination thereof;
   thereby treating an aging-associated condition in a subject.

By another aspect there is provided a pharmaceutical composition comprising a carrier and at least one of: an unmodified MSC; exosomes from an unmodified MSC; a dedifferentiated MSC; exosomes from a dedifferentiated MSC; a partially differentiated MSC; exosomes from a partially differentiated MSC and a combination thereof; for use in inhibiting aging.

As used herein the term “aging” refers to the natural deterioration over time of an organism, and specifically the cells of an organism. In some embodiments, aging comprises a diminished capacity of stem cells to produce differentiated cells. In some embodiments, aging comprises a diminished capacity of stem cells to self-renew. In some embodiments, aging comprises cells entering senescence. In some embodiments, aging comprises increased cell death. In some embodiments, aging comprises decreased cellular respiration. In some embodiments, aging comprises increased cellular reactive oxidation species (ROS). In some embodiments, aging comprises increased inflammation. In some embodiments, aging comprises increased fibrosis. In some embodiments, aging comprises increased scar tissue. In some embodiments, aging comprises cardiac heterotrophy. In some embodiments, aging comprises impaired glucose homeostasis. In some embodiments, aging comprises reduced cognitive function. In some embodiments, aging comprises reduced or impaired memory. In some embodiments, aging comprises reduced chondrocyte survival. In some embodiments, aging comprises increased levels of progerin, Serine/arginine-Rich Splicing Factor 1 (SRSF1) or both.

In some embodiments, aging is not skin aging. In some embodiments, aging is any type of aging except skin aging. In some embodiments, aging is muscle aging. In some embodiments, aging is not muscle aging. In some embodiments, aging is any aging except skin and muscle aging. In some embodiments, aging is neuronal aging. In some embodiments, aging is pancreatic aging. In some embodiments, aging is joint aging. In some embodiments, aging is brain aging. In some embodiments, aging is selected from muscle, neuronal, pancreatic and joint aging. In some embodiments, aging is selected from neuronal, pancreatic and joint aging.

In some embodiments, aging comprises decreased cognitive function. In some embodiments, aging comprises decreased muscle mass. In some embodiments, aging comprises decreased hormone production. In some embodiments, aging comprises impaired reflexes. In some embodiments, aging comprises impaired function of one of the systems of the body, including, but not limited to, the circulatory system, the muscular-skeletal system, the immune system, the respiratory system, the nervous system, the digestive system, the limbic system, glucose homeostatic system, neuro-muscular system, joint system and the renal system.

As used herein, an “aging-associated disease” refers to a disease of old age. In some embodiments, an aging-associated disease refers to a condition or disease whose prevalence increases with age. In some embodiments, an aging-associate disease is a disease that occurs with increasing frequency when there is increasing or increased cellular senescence.

In some embodiments, the aging associated disease is not a skin disease. In some embodiments, the aging associated disease is any aging disease that is not a skin disease. In some embodiments, the aging associated disease is selected from a muscular disease, a neuronal disease, a joint disease, and a pancreatic disease.

In some embodiments, the aging-associated disease is a muscular disease. In some embodiments, the aging-associated disease is not a muscular disease. In some embodiments, the muscular disease is selected from sarcopenia and fibrosis. In some embodiments, the muscular disease is selected from sarcopenia, cachexia and fibrosis. In some embodiments, the fibrosis is selected from: cardiac fibrosis, diaphragm fibrosis, and skeletal muscle fibrosis. In some embodiments, the fibrosis is cardiac fibrosis. In some embodiments, the fibrosis is skeletal muscle fibrosis. In some embodiments, the muscular disease comprises at least one of fibrosis, reduced muscle mass, and muscle atrophy.

In some embodiments, the aging-associated disease is a neurological disease. In some embodiments, the neurological disease is selected from: impaired memory, impaired cognitive function, dementia, stroke-related brain damage and Alzheimer's disease. In some embodiments, the neurological disease comprises impaired memory, impaired cognitive function or both. In some embodiments, the neurological disease is Alzheimer's.

In some embodiments, the aging-associated disease is a joint disease. In some embodiments, the joint disease is arthritis. In some embodiments, the joint disease is osteoarthritis. In some embodiments, the joint disease is disc degeneration.

In some embodiments, the aging-associated disease is a pancreatic disease. In some embodiments, the aging-associated disease is a disease of glucose homeostasis. In some embodiments, the aging-associated disease is type 2 diabetes.

In some embodiments, the aging-associated disease is Hutchinson-Gilford Progeria Syndrome (HGPS). In some embodiments, the methods of the invention are methods of treating HGPS.

In some embodiments, an aging-associated disease is selected from: sarcopenia, fibrosis, diabetes type 2, osteoarthritis, muscle atrophy, Alzheimer's disease, and HGPS. In some embodiments, an aging-associated disease is selected from: sarcopenia, fibrosis, diabetes type 2, arthritis, muscle atrophy, Alzheimer's disease, dementia, stroke-related brain damage and HGPS. In some embodiments, the aging-associated disease is cancer. In some embodiments, an aging-associated disease is selected from: sarcopenia, fibrosis, diabetes type 2, osteoarthritis, muscle atrophy, Alzheimer's disease, cancer and HGPS. In some embodiments, an aging-associated disease is selected from: sarcopenia, fibrosis, diabetes type 2, arthritis, muscle atrophy, Alzheimer's disease, dementia, stroke-related brain damage, cancer and HGPS. In some embodiments, an aging-associated disease is selected from: sarcopenia, dementia, vascular dementia, Alzheimer's disease, diabetes, cardiovascular disease, osteoporosis, progeroid syndromes, hypertension, arthritis, cataracts, kidney disease, liver disease, fibrosis and cancer. In some embodiments, the methods of the invention are for treating a first aging associated disease that is not cancer, and a second aging associated disease that is cancer. In some embodiments, the methods of the invention are for treating an aging associated disease that is not cancer and decreasing the risk of developing cancer.

In some embodiments, an aging-associated disease comprises diseases that display cellular damage similar to aging. In some embodiments, diseases that display damage similar to aging are selected from radiation-induced brain injuries, repetitive head injury syndrome, autism, ischemic injury, cerebral palsy, and HGPS. In some embodiments, ischemic injury is ischemic brain injury. In some embodiments, ischemic injury is ischemic heart injury. In some embodiments, an aging-associated disease is Hutchinson-Gilford Progeria Syndrome (HGPS).

In some embodiments, treating or inhibiting aging comprises at least one of: changing the microbiome in an aged subject, decreasing fibrosis, decreasing inflammation, decreasing inflammatory response in an aged subject, and decreasing production of reactive oxidation species (ROS). In some embodiments, treating or inhibiting aging comprises decreasing fibrosis. In some embodiments, treating or inhibiting aging comprises increasing muscle mass. In some embodiments, treating or inhibiting aging comprises increasing stem cell self-renewal. In some embodiments, the stem cells are neuronal stem cells. In some embodiments, the stem cells are satellite cells. In some embodiments, the stem cells are muscle stem cells. In some embodiments, treating or inhibiting aging comprises improving glucose homeostasis. In some embodiments, treating or inhibiting aging comprises increasing cognitive function. In some embodiments, treating or inhibiting aging comprises increasing memory. In some embodiments, treating or inhibiting aging comprises decreasing progerin levels, SRSF1 levels or both. In some embodiments, treating or inhibiting aging comprises increasing chondrocyte survival. In some embodiments, treating or inhibiting aging comprises treating cancer. In some embodiments, treating or inhibiting aging comprises reducing the risk of developing cancer.

As used herein, the term “mesenchymal stem cell” or “MSC”, refers to multipotent stromal stem cells that have the ability to differentiate into osteoblasts, adipocytes, myocytes, chondroblasts, skeletal muscle cells and endothelial cells. MSC are present in the bone marrow, adipose tissue, peripheral blood, chorionic placenta, amniotic placenta, umbilical cord blood, and dental pulp, among other tissues. The term “multipotent” refers to stem cells which are capable of giving rise to many cell types. In some embodiments, the unmodified MSC is derived from umbilical cord or chorionic placenta. In some embodiments, the unmodified MSC is derived from dental pulp, umbilical cord or chorionic placenta. In some embodiments, the unmodified MSC is derived from chorionic placenta. In some embodiments, the unmodified MSC is derived from umbilical cord. In some embodiments, the unmodified MSC is derived from dental pulp. In some embodiments, the unmodified MSC is derived from any one of umbilical cord, dental pulp and chorionic placenta. In some embodiments, the unmodified MSC is not derived from amniotic placenta. In some embodiments, the pharmaceutical composition is devoid of amniotic placenta MSCs. In some embodiments, the pharmaceutical composition is substantially devoid of amniotic placenta MSCs.

In some embodiments, the cell is a mammalian cell. In some embodiments, the cell is a human cell. In some embodiments, the cell is an animal cell such as of a veterinary animal. In some embodiments, the veterinary animal is selected from, a cat, a dog, a horse, a cow, a pig, a sheep and a goat. In some embodiments, the cell is a canine cell. In some embodiments, the cell is allogenic to a subject in need of treatment for a muscle-associated disease or muscle injury. In some embodiments, the cell is autologous to a subject in need of treatment for a muscle disease or a muscle injury. In some embodiments, the MSC is suspended in appropriate carrier for administration.

In some embodiments, the subject is a human. In some embodiments, the subject is a mammal. In some embodiments, the subject is a veterinary animal. In some embodiments, the subject is a dog/canine.

Chorionic, dental pulp and umbilical cord MSCs are well known in the art. In some embodiments, chorionic MSCs or their secreted vesicles can be identified by examining the expression of any of the following: a) one or more long non-coding RNAs (lncRNAs) selected from the group consisting of: SCAB, TU00176, LINC-VLDLR and optionally ROR; b) one or more miRNA selected form the group consisting of mir-3163, mir-128, mir-27a, mir-27b, mir-148a, mir-148b, mir-152, mir-651, mir-9, mir-466, mir-577, mir-380, mir-2909, mir-4803, mir-556-3p, mir-182, mir-4677-5p, mir-4672, mir-3942-5p, mir-4703-5p, mir-4765, mir-4291, mir-144, mir-1206, mir-4435, mir-452, mir-4676-3p, mir-25, mir-32, mir-363, mir-367, mir-92a, mir-92b, mir-340, mir-3620, mir-4324, mir-4789-5p, mir-346, mir-944, mir-3180-5p, mir-202, mir-511, mir-4326, mir-578, mir-4312, mir-4282, mir-597, mir-3689d, mir-2116, mir-4517, mir-199a-3p, mir-199b-3p, mir-3129-5p, mir-520d-5p, mir-524-5p, mir-203, mir-3942-3p, mir-501-5p, mir-143, mir-4770, mir-4422, mir-4495, mir-1271, mir-96, mir-1297, mir-26a, mir-26b, mir-4465, mir-4273, mir-1294, let-7a, let-7b, let-7c, let-7d, let-7e, let-7f, let-7g, let-7i, mir-4458, mir-4500, mir-98, mir-4652-3p, mir-4716-5p, mir-513a-5p, mir-223, mir-4288, mir-455-5p, mir-632, mir-4477b, mir-142-3p, mir-561, mir-4698, mir-3140-3p, mir-3662, mir-410, mir-376a, mir-376b, mir-1270, mir-620, mir-515-5p, mir-875-5p, mir-140-5p, mir-4256, mir-30a, mir-30b, mir-30c, mir-30d, mir-30e, mir-4254, mir-515-3p, mir-519e, mir-2964a-5p, mir-2115, mir-520a-5p, mir-525-5p, mir-1244, mir-3190, mir-548a-5p, mir-548ab, mir-548ak, mir-548b-5p, mir-548c-5p, mir-548d-5p, mir-548h, mir-548i, mir-548j, mir-548w, mir-548y, mir-559, mir-2681, mir-3671, mir-375, mir-4789-3p, mir-3143, mir-125a-5p, mir-125b, mir-4319, mir-5096, mir-338-5p, mir-493, mir-3153, mir-875-3p, mir-516a-3p, mir-323-3p, mir-3065-5p, mir-4762-3p, mir-3617, mir-641, mir-124, mir-506, mir-4531, mir-4512, mir-570, mir-4679, mir-3144-3p, mir-4777-3p, mir-4732-3p, mir-3177-5p, mir-548n, mir-4328, mir-2355-3p, mir-4330, mir-4524, mir-4719, mir-3976, mir-544, mir-3607-3p, mir-581, mir-205, mir-4731-3p, mir-4801, mir-3667-5p, mir-1245b-3p, mir-4760-3p, mir-137, mir-3194-3p, mir-342-3p, mir-2682, mir-449c, mir-532-3p, mir-4305, mir-1, mir-206, mir-613, mir-676, mir-1296, mir-196a, mir-196b, mir-3941, mir-4795-3p, mir-431, mir-607, mir-548k, mir-4464, mir-4748, mir-654-3p, mir-544b, mir-3074-5p, mir-3115, mir-4635, mir-4323, mir-548t, mir-4680-5p, mir-133a, mir-133b, mir-600, mir-1208, mir-4708-5p, mir-3123, mir-4251, mir-4307, mir-3185, mir-582-5p, mir-4436b-3p, mir-378, has, mir-378b, mir-378c, mir-378d, mir-378e, mir-378f, mir-378h, mir-378i, mir-422a, mir-4460, mir-200b, mir-200c, mir-429, mir-4470, mir, 1245b-5p, mir-3142, mir-576-3p, mir-548m, mir-4666-3p, mir-325, mir-330-3p, mir-3690, mir-548a-3p, mir-548e, mir-548f, mir-4709-5p, mir-532-5p, mir-539, mir-4303, mir-4302, mir-300, mir-381, mir-4645-3p, mir-3910, mir-1301, mir-5047, mir-188-5p, mir-3974, mir-3923, mir-3686, mir-670, mir-2052, mir-548a1, mir-3200-3p, mir-4686, has, mir-3545-5p, mir-194, mir-498, mir-3913-3p, mir-3168, mir-499-3p, mir-499a-3p, mir-656, mir-4762-5p, mir-4496, mir-141, mir-200a, mir-3529, mir-379, mir-3691-3p, mir-520f, mir-503, mir-4477a, mir-513a-3p, mir-3149, mir-3927, mir-1283, mir-4767, mir-487b, mir-4637, mir-19a, mir-19b, mir-4683, mir-548an, mir-1200, mir-4638-3p, mir-1825, mir-522, miR-24, miR-22-3p, miR-92, miR-378, miR-93; c) one of more secreted factors selected from the group consisting of HGF, wnt2, GDNF, Osteoprotegerin, MIP3a, NT-3, IL-6, IL-8, FGF7, NT-4, EGFL6 and optionally LIF and BDNF; d) one of more surface markers selected from: TCR alpha-beta, CD55, LIFR, and ST6GALNACS; e) one or more stemness and mesenchymal markers selected from: low YKL40 and KLF4; f) MSC-derived vesicle expression of one or more proteins selected from the group consisting of: COL4A2, LGALS3, SCUBE1, LGAS3, and S100A10; g) MSC-derived vesicle expression of one or more lncRNAs selected from the group consisting of BCMS, BIC, and optionally HAR1B; and h) a combination thereof.

In some embodiments, the chorionic MSCs may also be identified by cell-derived vesicles comprising one or more proteins selected from the group consisting of: CASK, COL3A1, B2M, CDH2, CTNNA1, DLG1, EGFR, F3, FARP1, GPC1, CDH2, CTNNA1, HAPLN1, LAMB1, LAMB2, LAMPC1, LGALS3BP, LOXL2, MCAM, NID1, OLXNB2, S100A6, TNC, WNT5A, and PLXNB2.

Other MSCs may be identified by markers such as are described in WO/2018083700, the content of which are herein incorporated by reference.

As used herein, the term “dedifferentiated MSC” refers to an MSC that has at least one increased stem cell characteristic, but still retains an MSC phenotype. In some embodiments, a de-differentiated MSC expresses at least one of SOX2, NANOG, OCT4 and KLF4. In some embodiments, a de-differentiated MSC expresses at least one of SOX2, NANOG, OCT4 and KLF4 at a level higher than it is expressed in an untreated MSC. In some embodiments, a de-differentiated MSC expresses a plurality of SOX2, NANOG, OCT4 and KLF4. In some embodiments, the dedifferentiated MSC is produced by introducing into an MSC at any one of NANOG, SOX2, KLF4, OCT4 and a combination thereof. In some embodiments, the introducing is ectopic or exogenous introducing. In some embodiments, the dedifferentiated MSC is produced by incubating an MSC in a medium containing 5-azacetidine (5-AZA). In some embodiments, the dedifferentiated MSC is produced by contacting an MSC with 5-AZA. In some embodiments, the dedifferentiated MSC is produced by incubating an MSC in acidic or hypoxic media. In some embodiments, the dedifferentiated MSC is produced by incubating an MSC with any one of 5-AZA, acidic media, hypoxic media and a combination thereof.

In some embodiments, an MSC phenotype comprises expression of at least one surface marker selected from the group consisting of: CD73, CD105, CD90, CD44 and CD146. In some embodiments, an MSC phenotype comprises expression of a plurality of surface markers selected from the group consisting of: CD73, CD105, CD90, CD44 and CD146. In some embodiments, an MSC phenotype comprises expression of IL-10. In some embodiments, an MSC phenotype comprises absence of Major Histocompatibility Complex protein II (MHCII) expression. In some embodiments, an MSC phenotype comprises at least one expression marker selected from the group consisting of: CD73, CD105, CD90, CD146, and CD44 expression and absence of MHCII expression. In some embodiments, an MSC phenotype comprises a plurality of expression markers selected from the group consisting of: CD73, CD105, CD90, CD146, and CD44 expression and absence of MHCII expression.

The term “expression” as used herein refers to the biosynthesis of a gene product, including the transcription and/or translation of said gene product. Thus, expression of a nucleic acid molecule may refer to transcription of the nucleic acid fragment (e.g., transcription resulting in mRNA or other functional RNA) and/or translation of RNA into a precursor or mature protein (polypeptide). In some embodiments, expression markers refer to RNA expression. In some embodiments, expression markers refer to protein expression. In some embodiments, surface expression markers refer to expression of proteins on the cell surface or in the plasma membrane of a cell.

In some embodiments, an MSC phenotype comprises anti-inflammation ability. In some embodiments, the MSC described herein is an anti-inflammatory cell. In some embodiments, an MSC phenotype comprises the ability to decrease inflammation. In some embodiments, an MSC phenotype comprises secretion of anti-inflammatory cytokines. Anti-inflammatory cytokines are well known to one of skill in the art, and include, but are not limited to, IL-10, IL-4, IL-13, and transforming growth factor beta (TGFβ).

In some embodiments, an MSC phenotype comprises the ability to home to sites of inflammation, injury or disease.

In some embodiments, an MSC phenotype comprises immunomodulation ability. In some embodiments, an MSC phenotype comprises the ability to modulate a subject's immune system.

In some embodiments, an MSC phenotype comprises immunosuppression ability. In some embodiments, an MSC phenotype comprises the ability to suppress a subject's immune system. In some embodiments, an MSC phenotype comprises the ability to decrease activation of T-cells.

In some embodiments, an MSC phenotype comprises the ability to home to sites of inflammation, injury or disease.

The term “differentiated MSC” refers to an MSC that have differentiated to possess a specific non-MSC phenotype and expresses markers of that phenotype, but also still retain an MSC phenotype. In some embodiments, a partially differentiated MSC is a cell of a mixed character with both an MSC phenotype and a phenotype of a different cell type. In some embodiments, the other cell type is selected from: a muscle cell, an astrocyte, a neuronal stem cell (NSC), and a differentiated neuron. In some embodiments, the muscle cell is selected from a satellite cell and a myoblast. In some embodiments, the differentiated neuron is a motor neuron. In some embodiments, the differentiated neuron is an oligodendrocyte.

Methods of differentiating MSCs are known in the art. In some embodiments, differentiation to an astrocyte phenotype is performed as described in US Application US20150037298. In some embodiments, differentiation to an NSC phenotype or a differentiated neuron phenotype is performed as described in US Application US20150037299. These cells and their secreted exosomes and vesicles increase synaptogenesis and cognitive function and enhance endogenous neural regeneration.

Differentiation of an MSC to a cell with a muscle phenotype can be achieved by any of the following protocols alone or in combination:

Protocol 1: In some embodiments, a cell of the invention can be produced by providing an MSC, contacting the MSC with at least one of an acidic medium, a ROCK inhibitor, and 5-AZA, introducing into the MSC HGF or PDGFβ, and introducing into the MSC PCAT1 and NEAT1.

Protocol 2: In some embodiments, a cell of the invention can be produced by providing an MSC, contacting the MSC with at least one of an acidic medium, a ROCK inhibitor, and 5-AZA, introducing in the MSC HGF or PDGFβ, and introducing into the MSC GAS5 and an inhibitor of PTENP1 expression.

Protocol 3: In some embodiments, a cell of the invention is produced by providing an MSC; contacting the MSC with at least one of: an acidic medium, a ROCK inhibitor, and 5-AZA, and introducing into the MSC at least one growth factor selected from the group comprising: PDGFAA, PDGFBB, EGF, VEGF, TGFβ, and IGF1.

Protocol 4: In some embodiments, a cell of the invention is produced by introducing into an MSC at least one transcription factor selected from the group consisting of: MYF5, PAX3, PAX7, dystrophin, microdystrophin, utrophin, MyoD and PAX3, MyoD and PAX7, and MyoD and MYF5.

Protocol 5: In some embodiments, a cell of the invention is produced by providing an MSC; contacting the MSC with at least one of an acidic medium, a ROCK inhibitor, and 5-AZA; and introducing into the MSC at least one long non-coding RNA (lncRNA) selected from the group consisting of: BIL, PAR5, BIC, DISC2, GAS5DLG2AS, 7SK, Y1, LINCRNA, PCAT-1 SFMBT2, Y4, SCAB, MALAT1, MEG3, NEAT1, EGO, GAS5, KRASP1, LOC28519, BC200, and H19. In some embodiments, the at least one lncRNA is selected from PAR5, DISC2 and PCAT1.

Protocol 6: In some embodiments, a cell of the invention is produced by providing an MSC; contacting the MSC with at least one of an acidic medium, a ROCK inhibitor, and 5-AZA; and introducing into the MSC at least one miRNA (miR) selected from the group consisting of: miR-10b, miR-22, miR-122, miR-125a, miR-140-5p, miR-143, miR-145, miR-146a, miR-148b, miR-150, miR-155, miR-181b, miR-215, miR-296, miR-330, miR-370, miR-429, miR-520, miR-524, miR-543, miR-550, miR-561, miR-564, miR-582, miR-583, miR-587, miR-613, miR-614, miR-629, miR-634, miR-645, miR-646, miR-649, miR-661, miR-662, miR-663, miR-665, miR-668, miR-671, miR-887, miR-1183, miR-1224, miR-1225, miR-1228, miR-1234, miR-1246, miR-1247, miR-1257, miR-1258, miR-1268, miR-1269, miR-1289, miR-1287, miR-1909, miR-1911, miR-759, miR-3150, miR-3174, miR-3180, miR-3191, miR-3197, miR-4292, miR-2115, miR-4312, miR-92, 93 and miR-99. In some embodiments, the at least one miR is selected from the group consisting of: miR-10b, miR-138, miR-154, miR-155, miR-181, miR-215, miR-614, miR-375, and miR-668. In some embodiments, the miR is selected from miR-143, miR-10a, miR-375, miR-1225 and a combination thereof. In some embodiments, miR-143, miR-10a, miR-375, miR-1225 are introduced.

Introduction of a gene, RNA, nucleic acid or protein into a live cell will be well known to one skilled in the art. As used herein, “introduction” refers to exogenous addition of a gene, protein or compound into a cell. It does not refer to increasing endogenous expression of a gene, protein or compound. Examples of such introduction include, but are not limited to transfection, lentiviral infection, nucleofection, or transduction. In some embodiments, the introduction is by transfection. In some embodiments, the introducing occurs ex vivo. In some embodiments, the introducing occurs in vivo. In some embodiments, the introducing occurs in vivo or ex vivo. In some embodiments, the introduction comprises introducing a vector comprising the gene of interest.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be a viral promoter.

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like. In some embodiments, the vector, miR, lncRNA or RNA inhibitory molecule are transfected into the MSC.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In one embodiment, plant expression vectors are used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

In some embodiments, introduction of a gene of interest comprises introduction of an inducible vector, wherein administration of a drug to the cell will induce expression of the gene of interest. Drug inducible vectors are well known in the art, some non-limiting examples include tamoxifen-inducible, tetracycline-inducible and doxycycline-inducible. In some embodiments, the inducible-vector is introduced to the MSC ex-vivo and the MSC is contacted with the inducing drug in-vivo. In this way expression of the induced gene, and as a result priming or differentiation of the MSC, only occurs in-vivo. In some embodiments, priming or differentiation of the MSC only occurs after the MSC has homed to a location in the body of a subject.

In some embodiments, introducing comprises introducing a modified mRNA. The term “modified mRNA” refers to a stable mRNA that maybe introduced into the cytoplasm of the cell and will there be translated to protein. Such a mRNA does not require transcription for protein expression and thus will more quickly produce protein and is subject to less regulation. Modified mRNAs are well known in the art.

In some embodiments, the unmodified MSC, dedifferentiated MSC or differentiated MSC expresses at least one anti-aging factor selected from: TIMP2, GDF11 and KLOTHO. In some embodiments, the unmodified MSC, dedifferentiated MSC or differentiated MSC expresses miR-675.

In some embodiments, TIMP2, GDF11, KLOTHO or miR-675 has been introduced into the MSC, dedifferentiated MSC or differentiated MSC. In some embodiments, inhibitors at least one of miR-29b and miR-34 have been introduced into the MSC, dedifferentiated MSC or differentiated MSC. In some embodiments, the inhibitor is an antagomir. In some embodiments, miR-375 has been introduced into the MSC. In some embodiments, the MSC expresses exogenous miR-375. In some embodiments, the MSC expresses exogenous kncRNA PLUTO. In some embodiments, miR-21 has been silenced in the MSC. In some embodiments, silencing comprises introducing into the cell an RNA inhibitory molecule. In some embodiments, the RNA inhibitory molecule binds to and inhibits the target miR. In some embodiments, the molecule is an antagomir. In some embodiments, a miR-21 antagomir has been introduced into the cell. In some embodiments, exogenous miR-375, lncRNA PLUTO, a miR-21 antagomir or a combination thereof has been introduced into the cell. In some embodiments, the MSC expresses exogenous miR-143. In some embodiments, the MSC expresses exogenous long non-coding RNA (lncRNA) MEG3. In some embodiments, the MSC expressed exogenous miR-143 and MEG3. In some embodiments, the MSC have been silenced for at least one of let-7, miR-424, 195, 16, 497, 135, 6793, 133b, 214 and 21. In some embodiments, the MSC expresses at least one exogenous miR selected from miR-10b, miR-138, miR-145 and miR-675. In some embodiments, the MSC expresses exogenous miR-145. In some embodiments, the MSC has been silenced for miR-154. In some embodiments, the MSC expressed exogenous miR-145 and has been silence for miR-154. In some embodiments, the MSC expressed at least one regulatory RNA selected from microRNA (miR)-10b, miR-138, miR-145, miR-375, miR-143, miR-675, lncRNA PLUTO and long non-coding RNA (lncRNA) MEG3. In some embodiments, the MSC expressed exogenous miR-143, miR-10a, miR-373 and miR-1225.

In some embodiments, a method of the invention comprises administration of a combination of cells and optionally their exosomes. In some embodiments, unmodified and dedifferentiated MSC are administered together. In some embodiments, unmodified and differentiated MSC are administered together. In some embodiments, differentiated and dedifferentiated MSC are administered together. In some embodiments, exosomes from any of these cell types are also administered together.

It will be understood by one skilled in the art that differentiated MSC will be differentiated to possess a phenotype of a cell relevant to the particular aspect of aging or aging related disease that is to be treated. For example, an MSC will be differentiated to have a muscle cell phenotype for the treatment of muscle loss, or a neuronal phenotype for the treatment of Alzheimer's disease. In some embodiments, MSCs differentiated to two different cell types are administered together. In some embodiments, an MSC differentiated to a muscle cell phenotype alone or with its exosomes is co-administered with an MSC differentiated to a neuronal phenotype alone or with its exosomes.

The term “extracellular vesicles”, as used herein, refers to all cell-derived vesicles secreted from MSCs including but not limited to exosomes and microvesicles. “Exosome”, as used herein, refers to cell-derived vesicles of endocytic origin, with a size of 50-100 nm, and secreted from MSCs. As a non-limiting embodiment, for the generation of exosomes cells are maintained with Opti-MEM and human serum albumin or 5% FBS that was depleted from exosomes. In some embodiments, exosomes comprise all extracellular vesicles.

“Microvesicles”, as used herein, refers to cell-derived vesicles originating from the plasma membrane, with a size of 100-1000 nm, and secreted from MSCs.

Exosomes, extracellular vesicles, or microvesicles can be obtained by growing MSCs in culture medium with serum depleted from exosomes or in serum-free media such as OptiMeM and subsequently isolating the exosomes by ultracentrifugation. Other methods associated with beads, columns, filters and antibodies are also employed. In some embodiments, the cells are grown in hypoxic conditions or incubated in medium with low pH so as to increase the yield of the exosomes. In other embodiments, the cells are exposed to radiation so as to increases exosome secretion and yield. In some embodiments, the exosomes are suspended in appropriate carrier for administration.

### Pharmaceutical Compositions

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein. In some embodiments, the pharmaceutical composition is devoid or substantially devoid of amniotic placenta MSCs. In some embodiments, the MSCs are in PBS, saline, or Ringer's solution.

### Additions to the Cells

In some embodiments, the unmodified, dedifferentiated, or differentiated MSC comprises at least one exogenous miR selected from: let7, miR-10b, miR-138, miR-145 and miR-675. In some embodiments, the unmodified, dedifferentiated, or differentiated MSC comprises exogenous let7, miR-10b, miR-138, miR-145 or miR-675. Each possibility represents a separate embodiment of the invention. In some embodiments, the unmodified, dedifferentiated, or differentiated MSC comprises silencing of miR-424, miR-195, miR-16, miR-497, miR-135, miR-6793, miR-21 or miR-133b. Each possibility represents a separate embodiment of the invention. In some embodiments, any of the exogenous miRs may be combined with any of the silencings. In some embodiments, the unmodified, dedifferentiated, or differentiated MSC comprises silencing of at least one of: miR-424, miR-195, miR-16, miR-497, miR-135, miR-6793, miR-21 and miR-133b.

In some embodiments, the unmodified, dedifferentiated, or differentiated MSC comprises exogenous let 7 and silencing of miR-133b. In some embodiments, the aging-associated disease is a muscular disease and the unmodified, dedifferentiated, or differentiated MSC comprises at least one exogenous miR selected from: let7, miR-10b, miR-138, miR-145 and miR-675. In some embodiments, the aging-associated disease is a muscular disease and the unmodified, dedifferentiated, or differentiated MSC comprises silencing of at least one of miR-424, miR-195, miR-16, miR-497, miR-135, miR-6793, miR-21 and miR-133b. In some embodiments, the aging-associated disease is a muscular disease and the unmodified, dedifferentiated, or differentiated MSC comprises exogenous let7 and silencing of miR-133b.

In some embodiments, the unmodified, dedifferentiated, or differentiated MSC comprises lncRNA PLUTO. In some embodiments, the unmodified, dedifferentiated, or differentiated MSC comprises miR-375. In some embodiments, the unmodified, dedifferentiated, or differentiated MSC comprises silenced miR-21. In some embodiments, the unmodified, dedifferentiated, or differentiated MSC comprises miR-375, lncRNA PLUTO, silenced miR-21, or a combination thereof. In some embodiments, the aging-associated disease is diabetes type 2, and the unmodified, dedifferentiated, or differentiated MSC comprises miR-375, lncRNA PLUTO, silenced miR-21, or a combination thereof. In some embodiments, the aging-associated disease is a neuronal disease, and the unmodified, dedifferentiated, or differentiated MSC comprises silenced miR-21. In some embodiments, the aging-associated disease is cancer or risk thereof, and the unmodified, dedifferentiated, or differentiated MSC comprises miR-375, silenced miR-21, or a combination thereof. In some embodiments, the aging-associated disease is diabetes type 2 and cancer or risk thereof, and the unmodified, dedifferentiated, or differentiated MSC comprises miR-375, lncRNA PLUTO, silenced miR-21, or a combination thereof. In some embodiments, the aging-associated disease is a neuronal disease and cancer or risk thereof, and the unmodified, dedifferentiated, or differentiated MSC comprises miR-375, lncRNA PLUTO, silenced miR-21, or a combination thereof. In some embodiments, the aging-associated disease is a neuronal disease and cancer or risk thereof, and the unmodified, dedifferentiated, or differentiated MSC comprises silenced miR-21.

In some embodiments, the unmodified, dedifferentiated, or differentiated MSC comprises MEG3. In some embodiments, the unmodified, dedifferentiated, or differentiated MSC comprises miR-143. In some embodiments, the unmodified, dedifferentiated, or differentiated MSC comprises MEG3, miR-143 or a combination thereof. In some embodiments, the aging-associated disease is osteoarthritis, and the unmodified, dedifferentiated, or differentiated MSC comprises MEG3. In some embodiments, the aging-associated disease is osteoarthritis, and the unmodified, dedifferentiated, or differentiated MSC comprises miR-143. In some embodiments, the aging-associated disease is a neuronal disease, and the unmodified, dedifferentiated, or differentiated MSC comprises MEG3, miR-143 or a combination thereof. In some embodiments, the aging-associated disease is cancer, and the unmodified, dedifferentiated, or differentiated MSC comprises MEG3, miR-143 or a combination thereof. In some embodiments, the aging-associated disease is osteoarthritis and cancer or risk thereof or a neuronal disease and cancer or a risk thereof, and the unmodified, dedifferentiated, or differentiated MSC comprises MEG3, miR-143 or a combination thereof.

In some embodiments, the unmodified, dedifferentiated, or differentiated MSC comprises exogenous miR-145. In some embodiments, the unmodified, dedifferentiated, or differentiated MSC comprises silencing of miR-154. In some embodiments, the unmodified, dedifferentiated, or differentiated MSC comprises exogenous miR-145, silencing of miR-154 or a combination thereof. In some embodiments, the aging-associated disease is a muscular disease, and the unmodified, dedifferentiated, or differentiated MSC comprises exogenous miR-145, silencing of miR-154 or a combination thereof.

In some embodiments, the unmodified, dedifferentiated, or differentiated MSC comprises exogenous miR-143, miR-10a, miR-373 and miR-1225. In some embodiments, the aging-associated disease is a muscular disease, and the unmodified, dedifferentiated, or differentiated MSC comprises exogenous miR-143, miR-10a, miR-373 and miR-1225. In some embodiments, the aging-associated disease is a neuronal disease, and the unmodified, dedifferentiated, or differentiated MSC comprises exogenous miR-143, miR-10a, miR-373 and miR-1225. In some embodiments, the aging-associated disease is HGPS, and the unmodified, dedifferentiated, or differentiated MSC comprises exogenous miR-143, miR-10a, miR-373 and miR-1225. In some embodiments, the aging-associated disease is any one of a muscular disease, a neuronal disease, HGPS, and a combination thereof and the unmodified, dedifferentiated, or differentiated MSC comprises exogenous miR-143, miR-10a, miR-373 and miR-1225.

In some embodiments, the unmodified, dedifferentiated, or differentiated MSC further comprise a targeting moiety on their cell surface or the surface of their exosomes. In some embodiments, the disease is a muscle disease and the targeting moiety is a muscle targeting moiety. In some embodiments, the moiety targets to a muscle cell selected from: a satellite cell, a smooth muscle cell, a skeletal muscle cell, and a cardiac muscle cell. In some embodiments, the disease is a neuronal disease and the targeting moiety is a neuron targeting moiety. In some embodiments, the moiety targets to a neuron selected from: an NSC, a motor neuron, a parasympathetic neuron, a GABAergic neuron, an astrocyte and a myelinated neuron. Targeting moieties are well known in the art, as are methods of expressing those moieties on a cells surface and a cell's extracellular vesicles.

In some embodiments, the unmodified, dedifferentiated, or differentiated MSC further comprise a therapeutic agent. In some embodiments, the therapeutic agent is a muscle therapeutic agent. In some embodiments, the therapeutic agent is a neuronal therapeutic agent. In some embodiments, the therapeutic agent is selected from the group consisting of: a drug, a read-through drug, an RNA, a DNA molecule, a vector, an exon skilling oligonucleotide, a microRNA (miR), a small interfering RNA (siRNA) an antagomir, a long noncoding RNA (lncRNA) and a virus.

In some embodiments, the drug is selected from oxytocin, melatonin, G-CSF, bortezomib and metformin.

In some embodiments, the methods of the invention are performed in conjunction with standard treatment of the disease or condition. In some embodiments, the methods of the invention further comprise administering an anti-aging drug. In some embodiments, the anti-aging drug is selected from the group consisting of: oxytocin, melatonin, G-CSF, and metformin.

By another aspect, there is provided a use of mitochondria derived from UC-MSCs and CH-MSCs to restore normal oxidative stress to an aged or diseased subject. In some embodiments, the use comprises restoring normal metabolism. In some embodiments, the use comprises restoring normal levels of ROS. In some embodiments, the use comprises restoring normal expression of at least one of: trophic factors, exosomes, and extracellular vesicles.

By another aspect, there is provided a method of restoring normal oxidative stress in a subject in need thereof, the method comprising administering to the subject a pharmaceutical composition comprising a carrier and a mitochondrion derived from UC or CH-MSCs.

### MSC Compositions

By another aspect there is provided an MSC expressing exogenous let-7 and an RNA inhibitory molecule that silences miR-133b. In some embodiments, the MSC is for use in treating muscle disease.

By another aspect there is provided an MSC expressing at least one exogenous miR selected from let7, miR-10b, miR-138, miR-145 and miR-675. In some embodiments, the MSC is for use in treating muscle disease. In some embodiments, the MSC further expresses at least one RNA inhibitory molecule that silences at least one of miR-424, miR-195, miR-16, miR-497, miR-135, miR-6793, miR-21, miR-154 and miR-133b.

By another aspect there is provided an MSC expressing at least one RNA inhibitory molecule that silences at least one of miR-424, miR-195, miR-16, miR-497, miR-135, miR-6793, miR-21, miR-154 and miR-133b. In some embodiments, the MSC is for use in treating muscle disease. In some embodiments, the MSC further expresses at least one exogenous miR selected from let7, miR-10b, miR-138, miR-145 and miR-675.

By another aspect there is provided an MSC expressing any one of exogenous miR-375, exogenous lncRNA PLUTO, an RNA inhibitory molecule that silences miR-21 and a combination thereof. In some embodiments, the MSC is for use in treating type 2 diabetes. In some embodiments, the MSC is for use in treating cancer. In some embodiments, the MSC expresses an RNA inhibitory molecule that binds to and inhibits miR-21 and is for use in treating neuronal aging. In some embodiments, the MSC is for use in treating the risk of developing cancer. In some embodiments, the MSC is for use in treating type 2 diabetes and cancer or the risk of developing cancer. In some embodiments, the MSC is for use in treating neuronal aging and cancer or the risk of developing cancer.

By another aspect there is provided an MSC expressing at least one of exogenous lncRNA MEG3, exogenous miR-143 and a combination thereof. In some embodiments, the MSC is for use in treating arthritis. In some embodiments, the arthritis is osteoarthritis. In some embodiments, the MSC is for use in treating neuronal disease. In some embodiments, the MSC is for use in treating cancer. In some embodiments, the MSC is for use in treating the risk of developing cancer. In some embodiments, the MSC is for use in treating arthritis and cancer or the risk of developing cancer. In some embodiments, the MSC is for use in treating neuronal disease and cancer or the risk of developing cancer.

By another aspect there is provided an MSC expressing any one of exogenous miR-145, an RNA inhibitory molecule that silences miR-154 and a combination thereof. In some embodiments, the MSC is for use in treating muscular disease/aging.

By another aspect there is provided an MSC expressing exogenous miR-143, miR-10a, miR-373 and miR-1225. In some embodiments, the MSC is for use in treating muscular disease/aging. In some embodiments, the MSC is for use in treating neuronal disease/aging. In some embodiments, the MSC is for use in treating HGPS. In some embodiments, the MSC is for use in treating any one of muscular disease/aging, neuronal disease/aging, HGPS and a combination thereof.

In some embodiments, the MSCs are genetically modified MSCs. In some embodiments, the MSCs are isolated.

By another aspect there if provided a pharmaceutical composition comprising a pharmaceutically acceptable carrier, adjuvant or excipient and at least one genetically modified MSC of the invention. It will be understood that the pharmaceutical compositions have the same uses as the cells that are in the composition.

As used herein, the terms “administering,” “administration,” and like terms refer to any method which, in sound medical practice, delivers a composition containing an active agent to a subject in such a manner as to provide a therapeutic effect. One aspect of the present subject matter provides for oral administration of a therapeutically effective amount of a composition of the present subject matter to a patient in need thereof. Other suitable routes of administration can include parenteral, subcutaneous, intravenous, intramuscular, intracranial, intranasal or intraperitoneal.

The dosage administered will be dependent upon the age, health, and weight of the recipient, kind of concurrent treatment, if any, frequency of treatment, and the nature of the effect desired.

The definitions of certain terms as used in this specification are provided herein. Unless defined otherwise, all technical and scientific terms used herein generally have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials described.

As used in this specification and the appended claims, the singular forms “a”, “an” and “the” include plural referents unless the content clearly dictates otherwise. For example, reference to “a nucleic acid” includes a combination of two or more nucleic acids, and the like.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the enumerated value.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### Examples

### Materials and Methods

### Preparation of Placenta and Umbilical Cord-Derived MSCs

Placenta and umbilical cord MSCs were isolated from humans, canines by the following protocol: The tissues were washed with PBS. The amniotic and the chorionic membrane were mechanically fragmented into small pieces and then submitted to enzymatic digestion in two stages. (1) Incubation with 0.25% trypsin/EDTA at 37° C. for 30 min in order to remove the epithelial cells. (2) Treatment with 0.1% collagenase IV for 60 min at 37° C. followed by inactivation with fetal calf serum. The cell suspension was then filtered through 100 μM filter and the centrifuged cells were seeded in 75 cm² Corning flasks in DMED medium/nutrient mixture F-12 (DMEM/F12) consisting of 15% fetal calf serum, 2 mM L-glutamine, 100 U/ml penicillin and 100 μg/ml streptomycin. Alternatively, cells were maintained in serum-free MSC medium. Similar procedures were employed for the preparation of MSCs from umbilical cord. The cells were then incubated with Rock inhibitor for 1 day followed by incubation in hypoxic conditions for additional 24 hr. The cells were maintained in medium deprived of exosomes.

### ROS Detection

Following treatment with myostatin cells were assayed for oxidative stress by use of the ROS-Glu H₂O₂ and GSH/GSSG-Glo assays (Promega). Mitochondrial membrane potential was measured using the JC-1 kit (Thermo).

### Exosome Isolation

Exosome isolation from cell culture media was performed at 4° C. by multi-step centrifugation. Briefly, media was centrifuged at 10,000×g for 30 minutes to remove large debris and then filtered through a 0.22 μm filter to remove small cell debris. The supernatant was then centrifuged at 100,000×g for 1-2 hours. Exosomes were identified by the expression of CD63, CD9 and ALIX by electron microscopy and by nanoparticle tracking analysis (NTA). Quantification of exosomes was analyzed by measuring the total protein concentration and by CD63 ELISA (SBI).

### qRT PCR

Total RNA was extracted using a RNeasy midi kit according to the manufacturer's instructions (Qiagen). Reverse transcription reaction was carried out using 2 μg total RNA. A primer optimization step was tested for each set of primers to determine the optimal primer concentrations. Primers, 25 μL of 2×SYBR Green Master Mix (Invitrogen), and 30 to 100 ng cDNA samples were resuspended in a total volume of 50 μL PCR amplification solution. The following primers were used: GDF11F: TCCGCCAGCCACAGAGCAAC; GDF11R: TCCAGTCCCAGCCGAAAGCC; TIMP2F: TGTGACTTCATCGTGCCCTG; TIMP2R: ATGTAGCACGGGATCATGGG; KLOTHOF: ACTCCCCCAGTCAGGTGGCGGTA; KLOTHOR: TGGGCCCGGGAAACCATTGCTGTC. Reactions were run on an ABI Prism 7000 Sequence Detection System (Applied Biosystems, Foster City, Calif.). Cycle threshold (Ct) values were obtained from the ABI 7000 software. S12 or B-actin levels were also determined for each RNA sample as controls.

### New Muscle Fiber Counts

Mice were injected with 25 μl of cardiotoxin in PBS into their TA muscle and sacrificed after 7 days. The muscle was dissected and stained for embryonic myosin heavy chain (MYH1), and cells positive for MYH1 with centrally located nuclei were scored as newly generated muscle. Alternatively, cells double positive for MyoD and Pax7 are considered asymmetrically dividing satellite cells and cells positive for NCAM are considered regenerating cells.

### Example 1: MSCs Express Anti-Aging Factors

MSCs from different sources have been demonstrated to have differential cellular effects and therapeutic impacts in various clinical models. In order to characterize different sources and subpopulations with specific characteristics and implications for more specific and efficient clinical applications, various parameters of these cells were compared and analyzed. MSCs from six sources were examined for factors that are known to possess anti-aging properties: bone marrow (BM), adipose (AD), amniotic placenta (AM), umbilical cord (UC), chorionic placenta (CH) and dental pulp (DP). Three specific factors stood out in the analysis: TIMP2, GDF11 and KLOTHO.

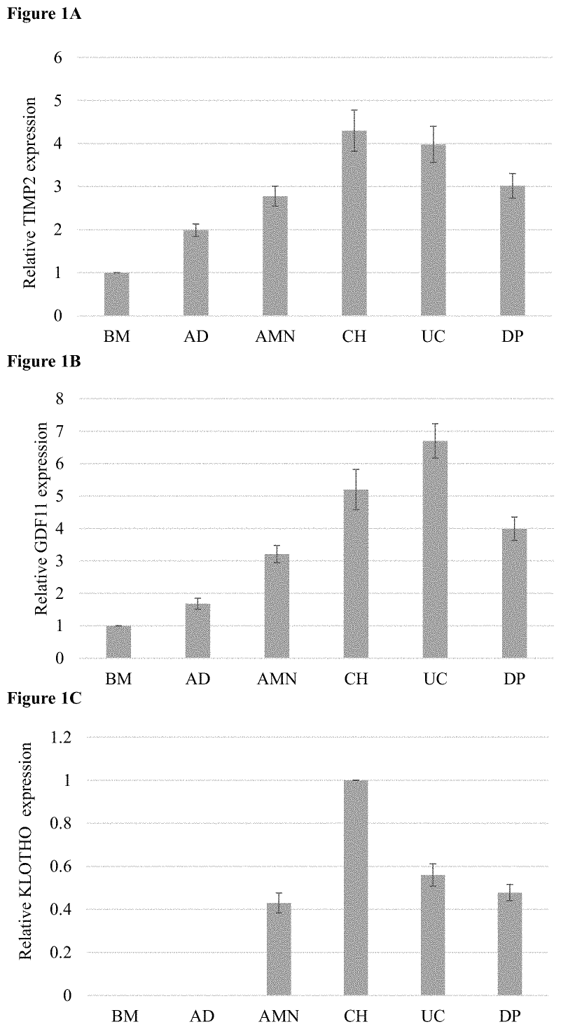

TIMP2 is a known longevity gene, that decreases with age, and has been implicated in improving cognitive function and neuronal health and plasticity. All six MSC types expressed TIMP2 to a degree, but the expression was lowest in BM-MSCs (FIG. 1A). AD, DP and AM expressed slightly higher TIMP2 levels, but the highest levels were observed in CH- and UC-MSCs, with MSCs derived from chorion having the highest expression levels.

GDF11's role as a longevity gene, is somewhat controversial, however, there is extensive data that GDF11 enhances muscle growth and regeneration and may also play a role in opposing skeletal, tendon and neuronal aging. Once again, all six MSC types expressed GDF11, with BM-MSC being the lowest expressing, followed by AD- AM- and DP-MSCs, and with CH- and UC-MSC once again having the highest expression (FIG. 1B).

KLOTHO is a well-known longevity gene, which has been implicated in reducing reactive oxidation species (ROS) and opposing age related decline in every cell type in which it is expressed. Unlike TIMP2 and GDF11, KLOTHO was not expressed in all the MSCs tested, but rather was completely absent from BM- and AD-MSCs (FIG. 1C). Moderate expression was observed in AM-, DP- and UC-MSCs, but CH-MSCs showed by far the highest expression, nearly twice that observed in the other three MSC types.

### Example 2: Chorionic and Umbilical Cord MSC Reduce ROS Production

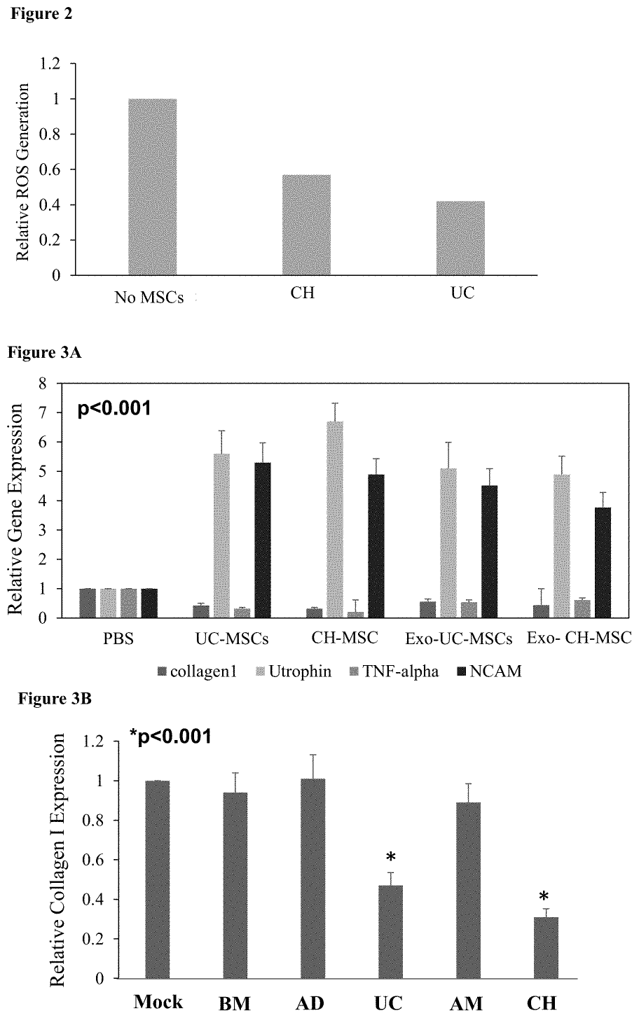

Myostatin is a known inhibitor of muscle growth and regeneration, and myostatin treatment is a model for muscle wasting and degeneration as it mirrors the increase in reactive oxidation species (ROS) produced in aging and damaged muscle. As KLOTHO is known to decrease ROS production, it was tested if CH- and UC-MSCs could also decrease the ROS production induced by myostatin. Human myoblasts were cultured in a 0.4 μM trans-well plate with UC- or CH-MSCs and treated with myostatin (40 ng/ml) for 3 days. Co-culture with CH-MSCs or UC-MSCs decreased ROS production by 43% or 58% respectively (FIG. 2). As the trans-well set up did not allow for cell-to-cell contact the MSC effect must be mediated by secreted factors, such as extracellular vesicles or possibly secreted KLOTHO.

### Example 3: MSCs Increase Muscle Regeneration and Decrease Fibrosis

Sarcopenia, the loss of muscle mass due to aging, could be treated in several ways, for example by increasing the regeneration rate of muscle tissue, or by replacing the lost muscle with new muscle cells. Additionally, as muscles age the risk of fibrosis, especially in cardiac muscle, increases greatly. MSCs from various tissues were injected (5×10⁵ cells per injection) into the quadriceps of mdx mice (a muscular dystrophy model) and expression of several key factors related to regeneration and fibrosis were examined after 4 weeks. Regeneration was monitored in the quadricep itself, while fibrosis was measured in the heart and diaphragm.

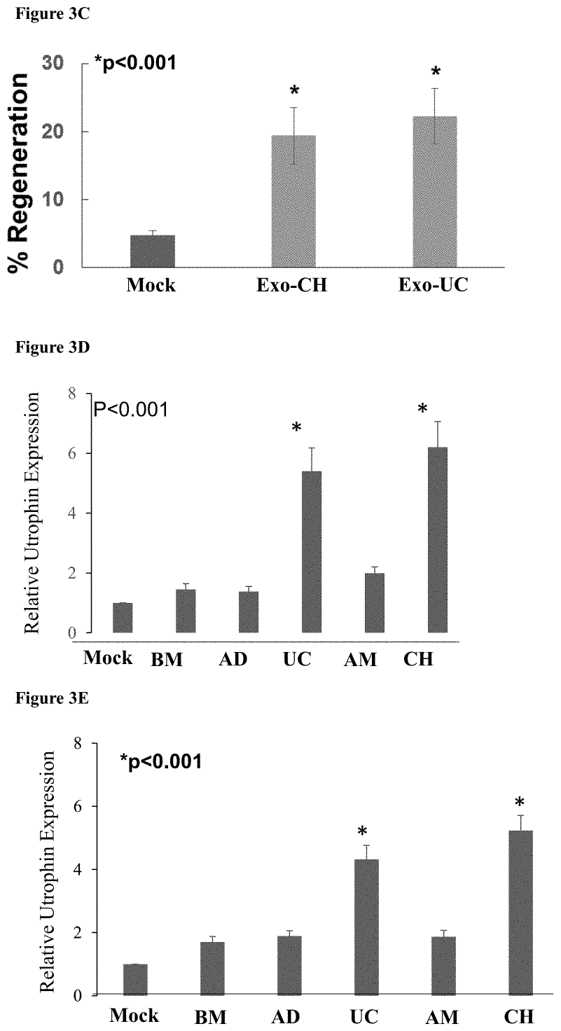

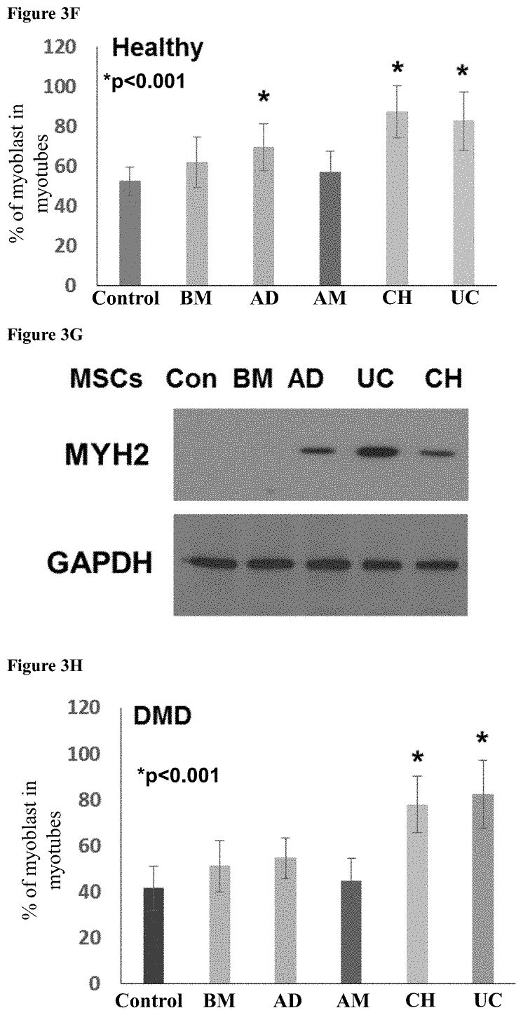

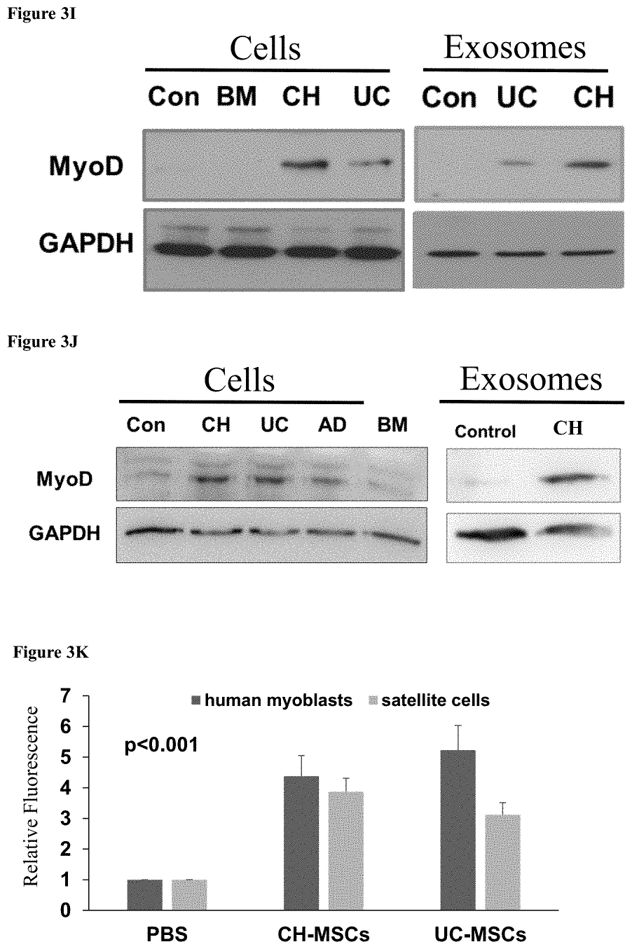

Both UC- and CH-MSCs induced a significant increase in the mRNA expression in the quadricep of two markers of cellular regeneration: embryonic myosin heavy chain (MYH1, fold increase, data not shown), and NCAM (FIG. 3A). Additionally, UC- and CH-MSCs significantly reduced the expression of Collagen I, a marker of fibrosis, in the diaphragm and the heart, while BM-, AD- and AM-MSCs had no effect (FIG. 3B). Lastly, both UC-MSCs and HC-MSCs decreased the expression of inflammatory markers such as TNFα and INFγ (FIG. 3A), while lower effects were observed with BM, AM and AD-derived MSCs. The same, although slightly reduced, gene expression changes were observed when only exosomes were injected (FIG. 3B), but still the amount of regeneration in the quadricep was significantly increased (FIG. 3C). Further, UC- and CH-MSCs significantly increased the expression of Utrophin (FIG. 3A, 3D), a protein that can functionally replace dystrophin. BM-, AD- and AM-MSCs caused only a very small not statistically significant increase in utrophin expression.

In vitro experiments with mouse cell line C2C12 and human muscle cells confirmed the expression changes caused by MSCs. Coculture in a trans-well plate of human muscle cells with MSCs showed that only UC- and CH-MSCs increased utrophin expression (FIG. 3E). Exosomes from these cells did as well. Muscle cell differentiation was also increased by AD-, CH-, and UC-MSCS and their exosomes, as measured by the formation of myotubes, (FIG. 3F) and expression of myosin heavy chain 2 respectively (FIG. 3G). However, when muscle cells from DMD patients were cocultured with MSCs only CH- and UC-MSC increased the formation of myotubes (FIG. 3H). Coculture of human satellite cells with UC- and CH-MSC, and their exosomes, increased asymmetric division (MyoD expression), although BM-MSCs did not (FIG. 3I). And coculture with C2C12 mouse muscle cells, showed similar results (FIG. 3J).

### Example 4: MSCs Increase the Efficacy of Muscle Cell Engraftment

MSCs do not express MHCII molecules on their cell surface and thus are well tolerated as transplant cells. Further, MSCs have an immunomodulatory effect on the transplantee that results in immunosuppression which further improves tolerance. It has been proposed that many muscular diseases, muscular dystrophies and muscle injury, could be treated with muscle cell replacement therapy, however, such therapies have proven difficult to achieve owing to rejection of the graft. It was thus tested whether MSCs (CH and UC), when included in the graft, could decrease rejection and increase the engraftment of foreign cells. Generally, and throughout the following experiments, CH or UC MSCs were always used as they showed the greatest therapeutic and myogenic potential. Other areas of the placenta besides chorionic and amniotic tissues were examined, such as the placental villi, but no region showed the therapeutic potential that chorion did.

MSCs were co-transplanted with human muscle cells labelled with a fluorescent red cell tracker into the tibialis anterior (TA) muscle of wild-type mice. After 2 weeks, the level of red fluorescence in the muscle was measured by microscopy, and both myoblast engraftment and satellite cell engraftment was examined. As compared to transplant without any MSCs, both UC- and CH-MSCs significantly increased the engraftment of myoblasts and satellite cells (FIG. 3K). It was observed that UC-MSC co-transplant resulted in a better engraftment of myoblasts, while CH-MSC co-transplant resulted in a better engraftment of satellite cells, although the differences were not statistically significant. Similar results were observed 4 weeks after transplant as well.

Transplant of human astrocytes and neural stem cells (NSCs) was also tested. UC-MSCs or CH-MSCs were co-transplanted intrathecally with fluorescent red labeled cells and red fluorescence in the spinal cord was measured after 2 weeks and after 4 weeks in separate experiments. The level of red fluorescent after transplant of astrocytes was 4.55 (±0.67) with UC-MSCs and 3.89 (±0.54) with CH-MSCs and similar results were found for transplant of NSCs (control fluorescence set to 1). These experiments were repeated in MDX mice as well as a rat model for Amyotrophic Lateral Sclerosis (ALS) and in all cases co-transplantation with MSCs was found to improve muscle cell engraftment.

### Example 5: MSCs Increase Satellite Cell Asymmetric Division in an Aging Muscle Model

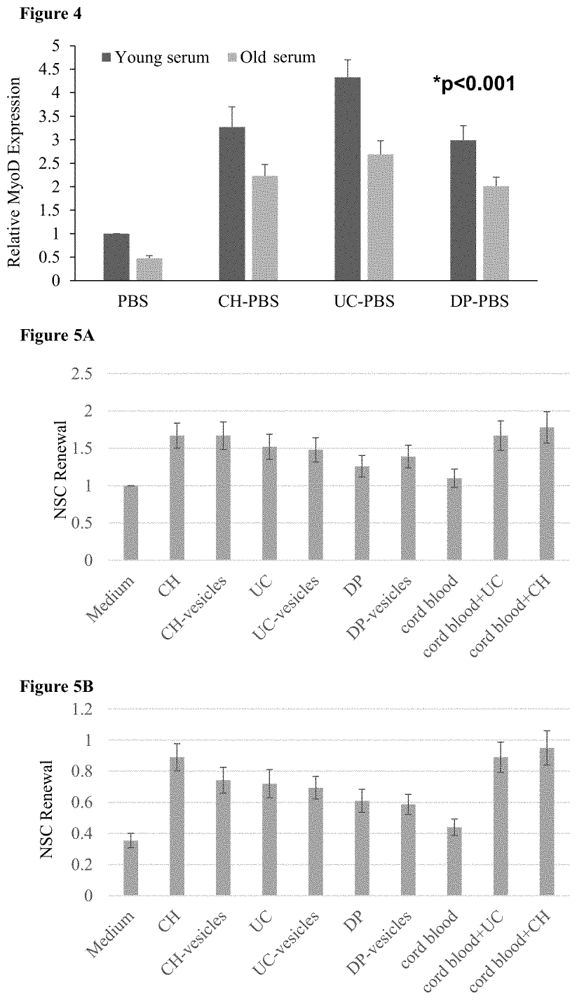

Muscle regeneration is mediated by a group of muscle stem-like cells known as satellite cells. These cells divide asymmetrically, producing a new satellite cell and a differentiated myoblast with every division. As muscles age satellite cells lose their proliferation and asymmetric division abilities. To test if the regeneration observed in muscle injected with MSCs, is due to increased satellite cell function, human satellite cells were incubated with serum from young (age 15-20) and old (age 55-60). Incubation with old serum, decreased the cells ability to divide asymmetrically into myoblasts (as measured by MyoD levels) by greater than 50% (FIG. 4). Trans-well coculture with CH-, DP- and UC-MSCs increased production of myoblasts by greater than 3, 3 and 4 times respectively. In the presence of MSCs, old serum still decreased the level of asymmetric division, but only by about 40 and 45%, and more importantly, the level of MyoD expression in the presence of MSC and old serum was still more than twice that of young serum with no MSCs. Exosomes from these MSCs added to culture had a similar, though reduced, effect on myoblast production. Similar effects were also observed when mouse C2C12 cells were used in place of human cells. Old serum (12-20 months) reduced MyoD expression as compared to young serum (3-4 months) and MSCs were able to reverse the effect and bring MyoD levels above even control levels. Thus, it would seem that the paracrine effects of UC-, DP- and CH-MSCs do indeed improve satellite cell function and ability to proliferate and differentiate and these MSCs therefore increase a satellite cell's ability to produce new muscle, even in the setting of advanced aging. Similar, but reduced effects were observed with AM-MSCs.

### Example 6: MSCs Increase Neuronal Stem Cell Self-Renewal in an Aging Model

The above described experiment using new and old serum was also performed using human neuronal stem cells (NSCs) in place of satellite cells. Doublecortin was used as a marker for NSC self-renewal, and just as had been observed for muscle, culture with old serum decreased the levels of doublecortin expression. Coculture with UC- and CH-MSCs and their exosomes also had the same effect, as they increased doublecortin expression, and even with old serum brought expression levels above that of NSCs grown alone with young serum.

To further model neuronal aging human neuronal stem cells (NSCs) were cultured with and without the addition of hydroxyurea. Hydroxyurea is a damaging agent that is a model for brain aging. NSCs were maintained in their regular growth condition, DMEM+EGF and FGF (10 ng/ml each) and were maintained as neurospheres. The cells were then dissociated and plated individually by serial dilution. Individual NSCs were grown per well and self-renewal capacity was measured by colony formation. The wells were in a transwell dish with either only medium or with CH-, DP- or UC-MSCs or their vesicles. MSCs or their exosomes increased the self-renewal of the NSCs, with the CH-MSCs having the strongest effect (FIG. 5A). Hydroxyurea reduced NSCs self-renew by 65%, while those low levels were more than doubled in the presence of the MSCs or their vesicles (FIG. 5B).

It has been reported that umbilical cord blood has advantages in treating brain conditions. As such the effect of cord blood was also tested. Cord blood alone was inferior to both types of MSCs and their vesicles, though combination of cord blood and the MSCs did have an additive effect (FIG. 5A-B).

### Example 7: Dedifferentiation of MSCs into a More Stem-Like Cell

MSCs are multipotent cells, but dedifferentiation of the cells into a more stem-like fate, could increase their ability to be used as a cell-replacement therapeutic in treating aging-related disorders and diseases. Various methods were employed to induce transient stem cell characteristics in MSCs, in order to increase their differentiation abilities in response to subsequent factors. Transfection of MSCs with a modified Nanog mRNA (such an mRNA is stable in the cytoplasm and can be immediately translated) prior to co-culture, increased overall stemness, as did a one-day incubation of MSCs with 5-azacytidine (5-AZA). The increase in stemness was enhanced when the two treatments were combined, with the transfected cells being incubated with 5-AZA. Further incubation in acidic media, or in hypoxia, further increased the dedifferentiation. Extracellular vesicles that were derived from these cells recapitulated the MSC effects and also were able to deliver some of these transcription factors to “aged” cells

MSCs that were primed to express a transient stem cell phenotype expressed SOX2, NANOG, OCT4 and KLF4 at levels higher than observed in untreated MSCs, as well as low levels of RTVO-1. However, these cells still expressed MSC markers CD73, CD105, CD90, CD146 and CD44 and did not express MHCII. Thus, while the cells had a greater differentiation potential they still exerted the beneficial paracrine effect that were observed in untreated MSCs. The cells were still non-immunogenic and had anti-inflammatory and immunosuppressive capabilities. Similar results were observed with the extracellular vesicles derived from these cells.

### Example 8: Dedifferentiated MSCs Improve Muscle Regeneration

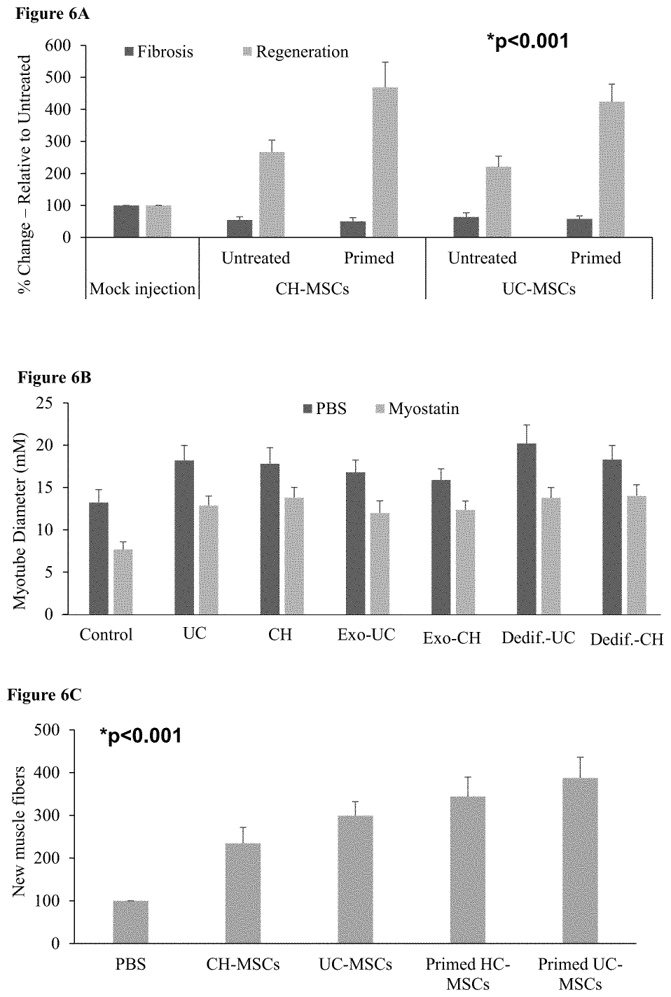

As dedifferentiated cells retain many of the characteristics of MSC, but also have increased differentiation potential, how they compare to untreated MSC in their ability to increase muscle regeneration and decrease fibrosis was tested. Untreated CH- and UC-MSCs were injected (5×10⁵ cells) into the left quadricep muscles of mdx mice, while dedifferentiated CH- and UC-MSCs were injected (5×105 cells differentiated by 5-AZA) into the right. As previously observed MSCs derived from both tissues decreased fibrosis in the diaphragm and the heart (Collagen I expression) and increased regeneration in the injected muscle (NCAM expression), but notably, dedifferentiated MSCs nearly doubled the level of regeneration, although the reduction in fibrosis was unchanged (FIG. 6A). Results were also observed when 0.5×10⁹ extracellular vesicles were injected to the quadricep.

### Example 9: Use of Dedifferentiated MSCs and Untreated MSCs to Treat Sarcopenia

Next the ability of untreated and dedifferentiated MSCs to ameliorate the muscle damage induced by myostatin was examined. Human myotubes were treated with myostatin (40 ng/ml) for 3 days and the diameter of the myotubes was monitored. Myostatin treatment mimics the effect of muscle atrophy and sarcopenia and the tubes were found to decrease in diameter by 42% on average (FIG. 6B, control). UC- and CH-MSC co-culture with the myotubules resulted in an average diameter decrease of only 29% and 22% respectively. Further, because co-culture actually increased the diameter of the myotubules when no myostatin was added, the reduction induced by myostatin only brought the diameter of the tubes to at, or just below, wild-type levels. Addition of exosomes from UC- and CH-MSC to the myotubes had a nearly identical effect as the MSCs themselves, as the diameter decrease was also 29% and 22% respectively. However, exosomes did not increase the diameter to quite the size that the MSCs had, and so after myostatin treatment the average diameter was slightly reduced. Lastly UC- and CH-MSC primed with 5-AZA or muscle coculture while having a similar % decrease had the largest final myotube diameter, as even after myostatin treatment the diameter of the myotubes was slightly increased as compared to the untreated control (FIG. 6B). This is likely due to primed MSCs merging into the myotubes.

The ability of untreated and dedifferentiated MSCs to induce new muscle formation in vivo was tested next. The tibialis anterior (TA) muscle of wild-type mice was injected with either PBS, CH-MSCs, UC-MSCs, dedifferentiated CH-MSCs or dedifferentiated UC-MSCs (5×105 cells for all) and then treated with cardiotoxin to induce muscle injury. At seven days the mice were sacrificed and newly generated muscle fibers in the gastrocnemius muscle were counted by noting MYH1 staining with centrally located nuclei. UC-MSCs more than doubled the number of new muscle cells, while CH-MSCs tripled it (FIG. 6C). Dedifferentiated MSCs had an even stronger effect as treated UC-MSCs or CH-MSCs increased the number of new muscle cells by 344% and 387% respectfully. Extracellular vesicles derived from these cells had similar effects. As UC derived vesicles (0.5×10⁹) caused a 2.38±0.345-fold increase and CH derived vesicles caused a 2.92±0.397-fold increase. Similarly, vesicles isolated from de-differentiated UC cells increased the number of new muscle cells by 3.24±0.42-fold and vesicles from de-differentiated CH by 3.59±0.419-fold.

In addition to measuring markers of an induced muscle cell phenotype, the expression of trophic factors GDNF, VEGF, CNTF and IGF1 was also examined. MSCs are known to express many trophic factors that support neuronal function. Loss of such expression would be an undesirable side effect of differentiation toward a muscle cell phenotype. Strikingly, not only was the expression of these four trophic factors retained in the hybrid cells (and primed cells as well), but in fact expression of all four was greatly increased over what is observed in untreated MSCs. This increase was strongest in hybrid cells derived from UC-MSCs, with an over 10-fold increase in VEGF expression, an over 6-fold increase in IGF1 expression, an over 5-fold increase in GDNF expression and an over 4-fold increase in CNTF expression. CH-MSCs also yielded a greater than 4-fold increase for all 3 factors. Similar results were observed whether the first or second differentiation protocol was performed.

These results taken as a whole suggest that MSCs reverse loss of muscle function and recovery ability associated with aging by improving/supporting both muscle and neural cells. Thus, MSC and MSC-extracellular vesicle-based therapies target both elements of the neuromuscular junction.

### Example 10: MSCs and their Secreted Exosomes have an Inhibitory Effect on Type 2 Diabetes

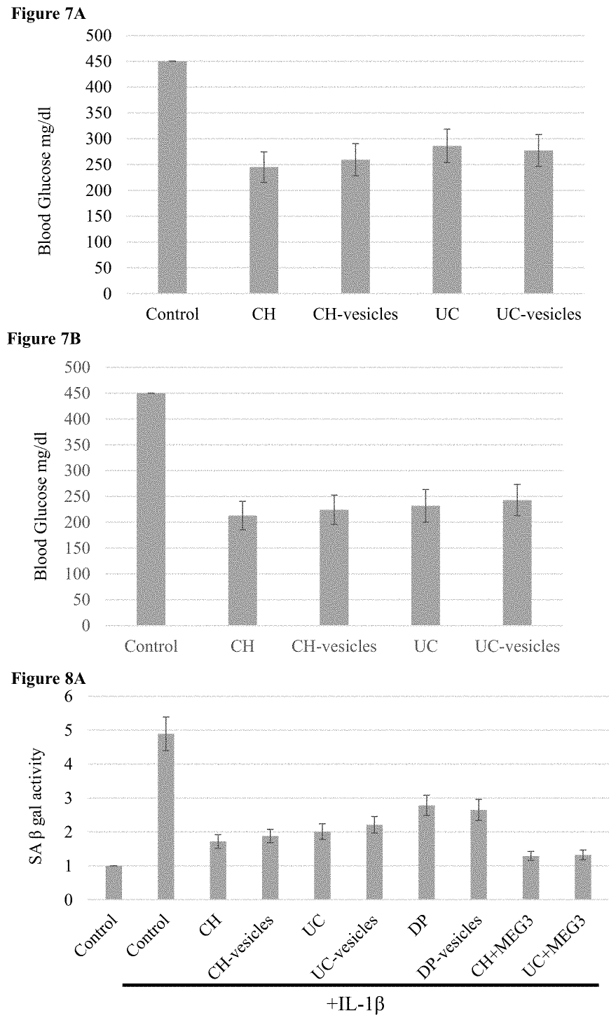

The incidence of type 2 diabetes increases with age. Unlike its juvenile counterpart Type 1 diabetes, aging in the pancreas is at least partially responsible for development of type 2 diabetes. To model type 2 diabetes, low-dose strezptocin treatment of mice, combined with a high fat diet was employed. These diabetic mice showed increased blood glucose levels when treated with control (PBS). The mice were administered CH- or UC-MSCs or their extracellular vesicles by intramuscular injection into the quadriceps muscle. Ten days after treatment of these mice decreased serum glucose levels were observed with all treatments (FIG. 7A). CH-MSCs were slightly superior to UC-MSCs (45% vs 35% reduction), as were their vesicles (42% vs 38% reduction).

In an effort to improve the effectiveness of the MSCs, the cells were made to ectopically express miR-375 and were silenced for miR-21. An even stronger effect on blood glucose levels was observed with these cells (FIG. 7B), with levels reduced by over 50% by CH-MSCs and their vesicles. MSCs were also made to overexpress the lncRNA PLUTO. CH-MSCs expressing PLUTO decreased glucose levels from 450 to 207±26.4 and their vesicles caused a decreased to 220±28.9. Similar results were observed with CH-MSCs and their vesicles expressing both miR-375 and PLUTO, and in this case the effect on glucose levels lasted longer. The PLUTO expressing MSCs were found to express insulin mRNA, which is one possible explanation for this extended effect.

### Example 11: MSCs and their Exosomes Treat Osteoarthritis

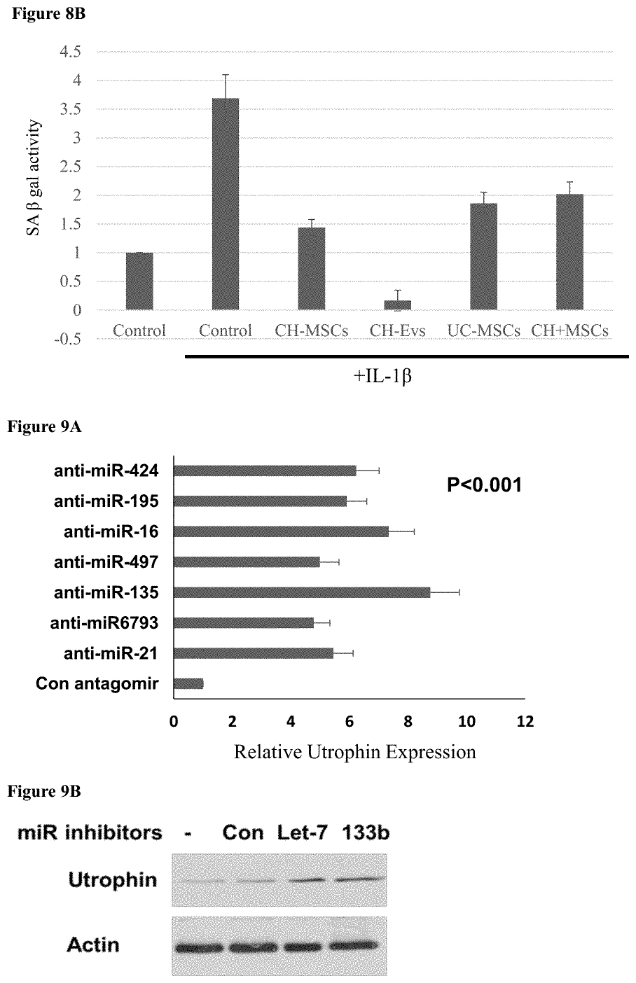

Osteoarthritis is the clinical syndrome manifested by joint pain and loss of joint form and function caused by the degeneration of articular cartilage. Chondrocyte aging is one of the hallmarks of this process. As such, IL-1 beta induced senesces in human chondrocytes was used as a model for testing the effect of MSCs on osteoarthritis. Human chondrocytes were grown in transwell culture and treated with 10 ng/ml IL-1 beta. After 5 days positive SA-beta-gal activity was measured as a proxy for senescence. Culture without other cells in the transwell was used as a control and was set to 1. Treatment with IL-1 beta increased senescence by a factor of five. MSCs and their vesicles reduced senescence nearly backed to untreated levels, with CH-MSCs and their vesicles having the strongest effect (FIG. 8A). MSCs were also transfected with the lncRNA MEG3 whose lose is known to induce osteoarthritis. Expression of MEG3 in the MSCs produced an even stronger reduction in senescence.

Osteoarthritis is also debilitating condition in the aging pet population. As such chondrocytes from canine were also tested. An experiment parallel to the above, was set up and human MSCs also greatly reduced senescence in the canine chondrocytes (FIG. 8B).

### Example 12: MSCs and their Exosomes Treat Cognitive Impairment

Unmodified UC- and CH-MSCs, as well as those differentiated to possess astrocyte, NSC, and neuronal phenotypes were found to have a protective effect against aging-associate mental conditions, such as Alzheimer's disease, progeria, and dementia. CH-MSCs and vesicles from those cells delayed development of dementia in an APP/PS1 (amyloid precursor protein/presenilinl) double transgenic mice model. Untreated mice develop amyloid-b deposits and plaques in brains at the age of 6-7 months and exhibit significant spatial learning/memory decline at about 8 months. Two assays were used to analyze the effectiveness of the MSC. In measuring recognition index, APP/PS1 mice demonstrated a decrease of 23.7% while CH-MSCs abrogated the decrease by 18.7% and their vesicles did so by 15.23%. In measuring platform crossing APP/PS1 mice demonstrated decreased crossing, as control mice averaged 4.56 crossings and the APP/PS1 mice averaged only 1.58 crossings. CH-MSCs treated mice increased to 3.23 crossings and those treated with vesicle improved to 2.99 crossings.

Radiation induced injury to the brain leads to profound and progressive impairments in cognitive function and has many similarities to neurodegenerative disorders, brain aging, stroke and repetitive head injury syndrome. We employed a mouse model of radiation to the brain and analyzed the cognitive function of mice (12 weeks old) that were treated with the following: PBS (control), CH-MSCs, CH-vesicles, CH-MSCs+miR-21 antagomir, and CH-vesicles+miR-21 antagomir. Mice were irradiated with 5Gy for 10 days. Two weeks later they mice were treated intracranially with MSCs or exosomes. 6 weeks after the treatment, the mice were trained to be familiar with the study environment for 2 weeks, then were examined for their ability to identify a novel object.

The index of identification is between 1 and −1. The smaller the number, the lower the ability to identify the novel object. It was found that irradiated mice treated with PBS had an identification index of −0.54. By contrast, mice treated with CH-MSCs had an index of 0.49. CH-vesicles had an index of 0.41; CH-MSCs+miR-21 antagomir had an index of 0.62 and CH-vesicles+miR-21 antagomir had an index of 0.67. Intranasal administration of the MSCs or their vesicles yielded similar results. Taken together, these results indicate that CH-MSCs and their vesicles can protect the brain from cognitive impairment associated with radiation induced injury (and by extension aging and trauma).

Radiation also decreased the ability of NSCs to proliferate by 39.7%. CH-MSCs and CH-vesicles however, increased this proliferation by 69.4% and 62.3% respectively. MSCs with miR-21 antagomir and their vesicles had an even greater effect: CH-MSCs+miR-21 antagomir increased proliferation by 84.5% and CH-vesicles+miR-21 antagomir increased proliferation by 81.5%. Since this model is informative for neurodegenerative diseases, brain aging, stroke, repetitive brain injury syndrome and vascular dementia, these MSCs and their vesicles can be beneficial for these conditions as well.

### Example 13: Differentiated MSCs Improve Cellular Function and Act as Cellular Replacements

MSCs can be differentiated into astrocyte-like, neuronal stem cell (NSC)-like, motor neuron-like, and muscle cell-like cells. However, even after this partial differentiation the cells still retain an MSC like character. This allows the cells to still home to sites of damage and disease, and to still exert anti-inflammatory and immunomodulatory effects. However, these cells have the added benefit of being able to act as therapeutic replacements for damages or aged cells and tissues.

Hutchinson-Gilford Progeria Syndrome (HGPS) is a rare genetic disorder that causes systemic premature aging in children. This is due to mutation in a gene called LMNA that encoded a truncated and toxic protein lamin A, also called progerin. The production of the toxic protein is also impacted by an RNA-binding protein called SRSF1. CH-MSCs, and vesicles from them, decreased progerin expression in fibroblasts derived from HGPS patients by 34.1% and 36.9%, respectively. Further Ch-MSCs and their vesicles decreased the expression of SRSF1 as well by 49.6% and 52.4%, respectively.

MSCs, especially those from UC and CH, when differentiated to possess satellite cell and myoblast phenotypes were found to have added therapeutic benefit in treating muscle aging, sarcopenia and muscle fibrosis. This added benefit is likely due to the cells actively merging with the preexisting muscle syncytium and serving as a cell preplacement therapy in addition to the MSC paracrine effects.

### Example 14: Over-Expression of RNA Therapeutics in the MSCs

Several potentially promising therapeutics exist for muscle, motor neuron, and peripheral neuron disease and injury. However, frequently there is difficulty in delivering the therapeutic directly and specifically to the injured or diseased area. Due to the homing ability of MSCs, along with their large repertoire of secreted vesicles it was hypothesized that MSCs might serve as ideal delivery agents to muscles and neurons.

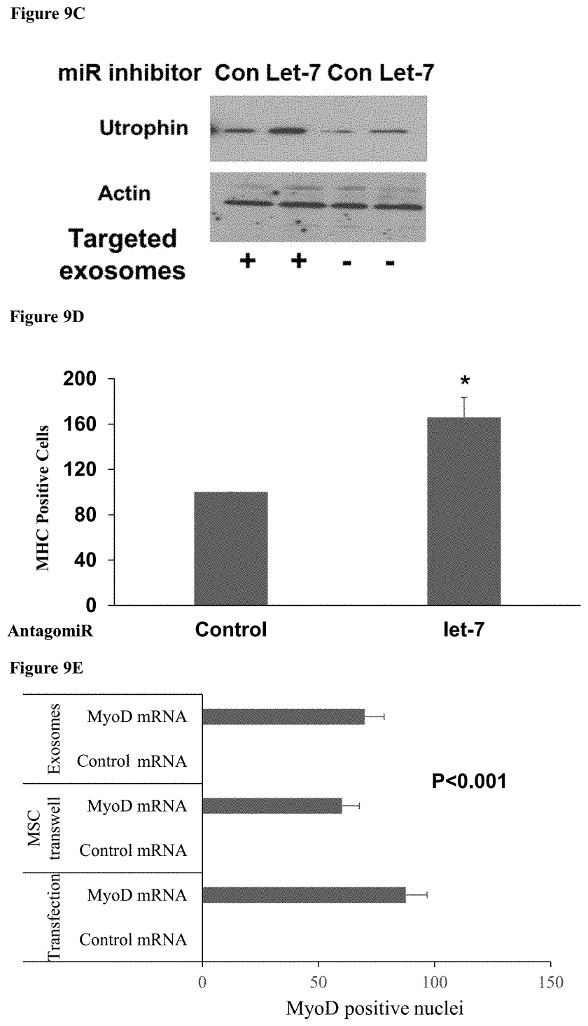

To test this hypothesis exosomes derived from unmodified CH- and UC-MSCs were loaded with antagomirs against several microRNAs (miRs) known to reduce utrophin expression (anti-miR-424, 195, 16, 497, 135, 6793, and 21), and incubated with human myoblasts. Incubation with these exosomes greatly increased utrophin mRNA expression in the myoblasts, indicating that the exosomes had successfully transferred the antagomirs to the myoblasts (FIG. 9A).

Similarly, CH-MSCs transfected with a let-7 antagomir or a miR-133b antagomir transferred the antagomir to muscle cells in vitro resulting in increased utrophin protein expression (FIG. 9B). Exosomes from these cells also successfully increased utrophin protein expression in vitro (FIG. 9C). Next, exosomes targeted to muscle cells by a M-cadherin epitope on the exosome surface were administered to a mixed muscle cell/astrocyte culture. The targeted-exosomes containing anti-let-7 increased utrophin expression in 55-68% of the astrocyte cells in the culture, but also did so in 85-92% of muscle cells in the culture (FIG. 9C, muscle cell lysate shown). This indicated that not only do the exosomes transfer antagomirs, but that muscle targeting moieties on the exosomes (or MSCs) will increase the effectiveness of the transfer. Let-7 is also known to decrease myosin heavy chain expression and thus inhibit muscle regeneration. The let-7 antagomir also significantly increased myosin heavy chain expression (FIG. 9D) and thus had a double therapeutic benefit.

Delivery of dystrophin protein is also a much-investigated therapeutic avenue, however, recombinant dystrophin induces a robust immunogenic response and, as yet, no effective delivery system has been discovered. As MSCs have immunosuppressive abilities, unmodified CH-MSCs were infected with viral vectors expressing dystrophin and microdystrophin in hopes that they would allow for dystrophin expression without an immune response. To further augment the effects of these plasmids, MSCs expressing an antagomir to miR-214, a miR that targets dystrophin, were also employed. The combined effect of the dystrophin plasmid and anti-miR-214, were striking with dystrophin expression increased by about 4.5-fold. Importantly, this treatment also increased utrophin expression. Thus, anti-miR-214 delivery also has a double therapeutic benefit as it increases both dystrophin and utrophin expression.

The ability of exosomes and MSC to deliver therapeutics was further tested by loading MSCs and exosomes with a modified myoD mRNA. Such an mRNA upon entering the cytoplasm of a cell can be immediately translated into protein. Direct addition of myoD loaded exosomes to myoblasts as well as coculture of loaded MSCs and myoblasts in a trans-well plate resulted in robust myoD expression as measured by the number of myoD positive nuclei (FIG. 9E). Direct transfection of cells with the modified myoD mRNA was used as a positive control, and indeed transmission of the mRNA by exosome or coculture was nearly as effective as direct administration by transfection. Thus, MSCs and their exosomes can effectively deliver modified mRNAs, similar to other RNA molecules.

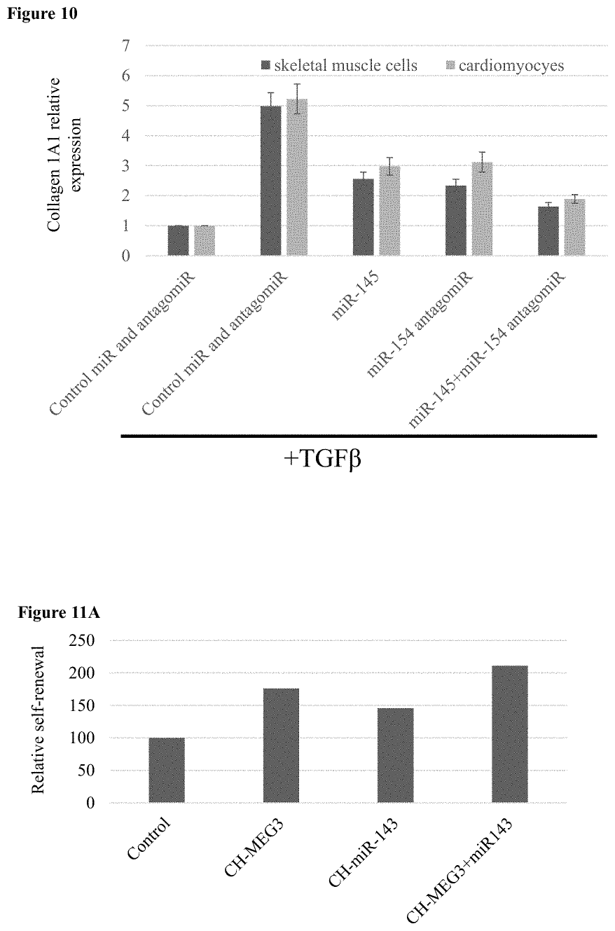

CH-MSCs were also transfected with miR-145 and an antagomir to miR-154, and the cells ability to treat fibrosis was monitored. Fibrosis in various muscles in the body is a hallmark of advanced aging. TGF-beta induced fibrosis was used as a model. Skeletal muscle cells and cardiomyocytes were grown in transwell culture and treated with 30 ng/ml TGF-beta. After 3 days the expression of collagen 1A1 was monitored by RT-PCR. TGF-beta increased collagen expression by about 5-fold in both muscle cells in the presence of non-modified MSCs, however MSCs expressing miR-145, antagomir-154 and a combination of the two profoundly reduced fibrosis (FIG. 10). Similar results were observed with extracellular vesicles derived from the CH-MSCs expressing miR-145 and anti-miR-154. UC-MScs and their secreted exosomes also decreased tissue fibrosis when loaded with miR-145 and miR-154 antagomir, however their effect was reduced.

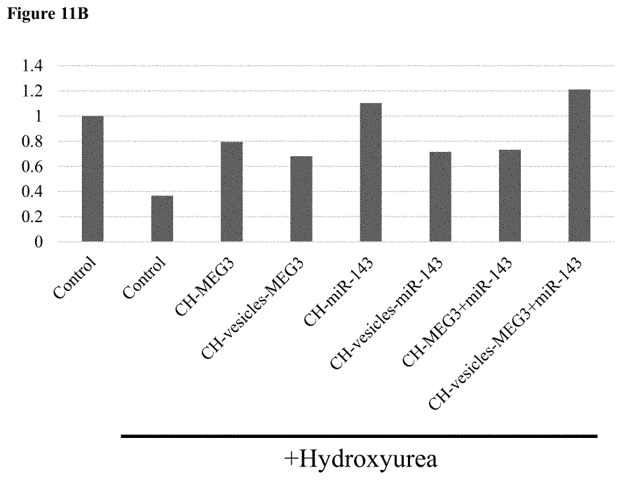

As previously described MEG3 expression is beneficial in osteoarthritis, but it also has a role in neuronal self-renewal. Similar to previous experiments with NSC renewal, NSCs were grown in a transwell with CH-MSCs expressing MEG3, miR-143 or a combination of the two. Each MSC increased NSC self-renewal, with the combination of the two more than doubling the self-renewal (FIG. 11A). Similarly, these MSCs and their vesicles also increased the self-renewal of NSCs treating with hydroxyurea which represents a model of neural aging (FIG. 11B).

Lastly, CH-MSCs were transfected with miR-143, miR-10a, miR-373 and miR-1225. These cells were cocultured with satellite cells in a transwell dish and were found to increase satellite cell proliferation and differentiation by 5.2-fold and 3.78-fold respectively. These same cells also increased NSC self-renewal after hydroxyurea treatment by 4.12-fold. Further, they decreased progerin expression in fibroblasts from HGPS patients by 42.1%. Lastly, these genetically modified MSCs also exerted anti-rumor effects in cancer cell lines, primary cancer cells and cancer stem cells.

### Example 15: MSCs and their Exosomes Exert Multi-Tumor Effect

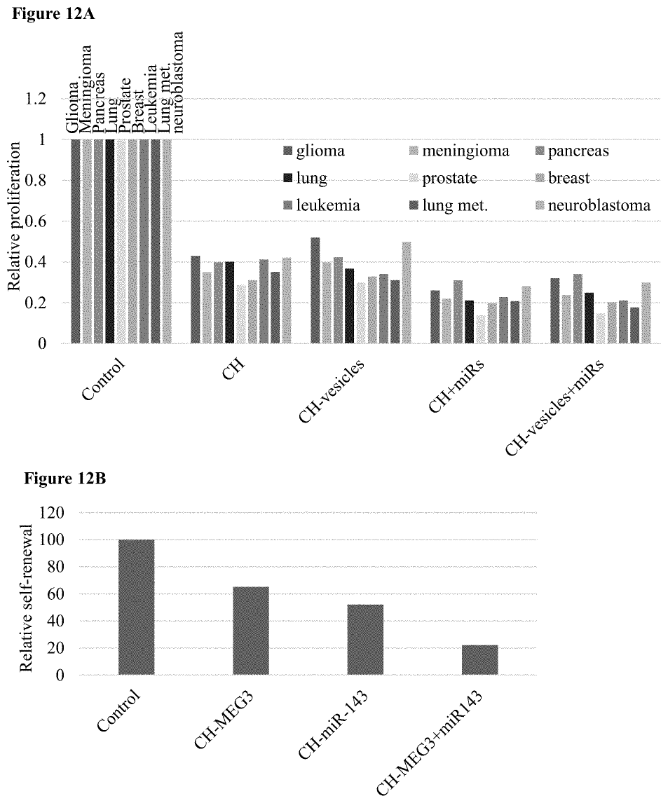

Aging is characterized by the increased incidence of tumor occurrence. Thus, any anti-aging treatment should be verified not to be tumor promoting or better to have anti-tumor effects. CH-MSCs and their extracellular vesicles were tested for their effect on nine different cancers (glioma, meningioma, pancreatic, lung, prostate, breast, leukemia, lung metastasis and neuroblastoma) (FIG. 12A). In all the cancers tested the MSCs and their vesicles has a strong inhibitory effect on cancer cell proliferation. Moreover, MSCs and vesicles derived from cells that overexpress miR-375 and were silenced for miR-21 exerted a stronger anti-tumor effect. Similar results were obtained with both UC-MSCs and their vesicles. CH-MSCs expressing exogenous MEG3 and miR-143 were also tested for their ability to inhibit self-renewal of lung tumor stem cells. The experiments were similar to those performed with NSCs, only in this case the MSCs inhibited tumor stem cell self-renewal with the combination of MEG3 and miR-143 producing a reduction of also 80% (FIG. 12B).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of inhibiting aging or treating an aging-associated disease in a subject, the method comprising administering to the subject a pharmaceutical composition substantially devoid of amniotic placenta mesenchymal stem cells (MSCs), and comprising a pharmaceutically acceptable carrier and at least one of:
a. a chorionic placenta MSC;
b. exosomes from a chorionic placenta MSC;
c. a dedifferentiated MSC;
d. exosomes from a dedifferentiated MSC;
e. a differentiated MSC;
f. exosomes from a differentiated MSC and
g. a combination thereof;
thereby inhibiting aging in a subject.

2. The method of claim 1, wherein said dedifferentiated MSC is produced by introducing into an MSC any one of NANOG, SOX2, KLF4, OCT4 and a combination thereof.

3. The method of claim 1, wherein said dedifferentiated MSC is produced by incubating an MSC in a medium containing 5-azacetidine (5-AZA) and optionally further incubating said MSC in an acidic medium or in a hypoxic medium.

4. The method of claim 1, wherein said aging is selected from muscle aging, neuronal aging, pancreatic aging and joint aging.

5. The method of claim 4, wherein neuronal aging comprises impaired cognitive function, impaired memory or both.

6. The method of claim 4, wherein muscle aging comprises reduced muscle mass, increased fibrosis or both.

7. The method of claim 1, wherein said aging associated disease is selected from sarcopenia, fibrosis, diabetes type 2, arthritis, muscle atrophy, Alzheimer's disease, dementia, stroke-related brain damage, and Hutchinson-Gilford Progeria Syndrome (HGPS).

8. The method of claim 7, wherein said fibrosis is cardiac fibrosis or skeletal muscle fibrosis.

9. The method of claim 7, wherein said arthritis is osteoarthritis.

10. The method of claim 1, wherein inhibiting aging comprises at least one of: decreasing fibrosis, decreasing inflammation, decreasing production of reactive oxidation species (ROS), increasing muscle mass, increasing stem cell self-renewal, improving glucose homeostasis, increasing cognitive function, increasing memory, increasing chondrocyte survival and decreasing levels of progerin, SRSF1 or both.

11. The method of claim 10, wherein said stem cell is any one of a neuronal stem cell (NSC) and a satellite cell.

12. The method of claim 1, wherein said treating comprises
a. treating an aging associated disease that is not cancer; and
b. reducing the risk of developing cancer, treating cancer or both.

13. The method of claim 1, wherein said differentiated MSC is differentiated toward any one of an astrocyte, a neural stem cell, a motor neuron, an oligodendrocyte, a satellite cell and a myoblast.

14. The method of claim 1, further comprising introducing into said MSC, dedifferentiated MSC or differentiated MSC at least one regulator RNA selected from: microRNA (miR)-10b, miR-10a, miR-138, miR-145, miR-373, miR-1225, miR-375, miR-143, miR-675, long non-coding RNA (lncRNA) MEG3 and lncRNA PLUTO.

15. The method of claim 1, further comprising introducing into said MSC, dedifferentiated MSC or differentiated MSC at least one RNA inhibitory molecule that binds to and inhibits at least one of let-7, miR-424, miR-195, miR-16, miR-497, miR-135, miR-6793, miR-133b, miR-214, miR-154 and miR-21.

16. The method of claim 1, wherein said subject is a human.

17. The method claim 1, wherein said subject is a veterinary animal.

18. A genetically modified MSC, said MSC comprising any one of:
(i) an exogenous microRNA let-7 and an RNA inhibitory molecule that binds to and inhibits miR-133b;
(ii) at least one exogenous miR selected from miR-10b, miR-138, miR-145 and miR-675,
(iii) at least one RNA inhibitory molecule that binds to and inhibits at least one of miR-424, miR-195, miR-16, miR-497, miR-135, miR-6793, miR-21 and miR-133b;
(iv) at least one of exogenous miR-145, an RNA inhibitory molecule that binds to an inhibits miR-154 and a combination thereof;
(v) at least one of exogenous miR-145, an RNA inhibitory molecule that binds to an inhibits miR-154 and a combination thereof;
(vi) exogenous miR-375, exogenous lncRNA PLUTO, an RNA inhibitory molecule that binds to and inhibits miR-21 and a combination thereof;
(vii) at least one of: exogenous lncRNA MEG3, exogenous miR-143 and a combination thereof; and
(vii) MSC comprising exogenous miR-143, miR-10a, miR-373 and miR-122S.

19. (canceled)

20. (canceled)

21. (canceled)

22. (canceled)

23. (canceled)

24. (canceled)

25. A pharmaceutical composition, comprising
a. the genetically modified MSC of claim 18; and
b. a pharmaceutically acceptable carrier, adjuvant or excipient.

26. (canceled)

27. (canceled)

28. (canceled)

29. (canceled)

30. (canceled)

31. (canceled)
